(19) United States
(12) Patent Application Publication
(10) Pub. No.: US 2026/0002945 A1
(43) Pub. Date: Jan. 1, 2026

(54) METHODS OF TREATING, AMELIORATING AND/OR PREVENTING WOUNDS

(71) Applicant: Drexel University, Philadelphia, PA (US)

(72) Inventors: Kara L. Spiller, Glenside, PA (US); Jessica M. Eager, Philadelphia, PA (US)

(21) Appl. No.: 18/880,770

(22) PCT Filed: Jul. 7, 2023
(86) PCT No.: PCT/US23/27066

§ 371 Date: Jan. 2, 2025

Related U.S. Application Data
(60) Provisional application No. 63/359,360, filed on Jul. 8, 2022.

(51) Int. Cl. G01N 33/68; A61K 35/50; A61K 45/06; A61P 1/04

(52) U.S. Cl. CPC G01N 33/6893; A61K 35/50; A61K 45/06; A61P 1/04; G01N 2333/523; G01N 2800/062; G01N 2800/52; G01N 2800/60

(57) ABSTRACT

Described herein is method of treating, ameliorating and/or preventing a wound in a subject in need thereof. The method includes: collecting a first sample from the wound at a first timepoint and determining a first CCL1 (chemokine ligand 1) level in the first sample; administering to the subject a first treatment that promotes wound healing; collecting a second sample from the wound at a second timepoint after the first timepoint and determining a second CCL1 level in the second sample; and (a) if the second CCL1 level is higher than the first CCL1 level times a predetermined value, continuing the administration of the first treatment or discontinuing the first treatment, (b) if the second CCL1 level is equal to or lower than the CCL1 level times the predetermined value, administering to the subject a second treatment that promotes wound healing.

### CROSS-REFERENCE TO RELATED APPLICATIONS

The present application claims priority under 35 U.S.C. § 119(e) to U.S. Provisional Patent Application No. 63/359,360, filed Jul. 8, 2022, which is incorporated herein by reference in its entirety.

### BACKGROUND

Wound healing is a dynamic process that are considered to occur in four phases: hemostasis, inflammation, proliferation, and remodeling. Chronic wounds—wounds without significant progress toward healing after prolonged time—are often trapped at one of these four phases, often the inflammation phase.

Diabetic foot ulcers (DFUs) is a type of chronic wound. DFUs are a major complication for diabetic patients. They occur in approximately 15% of patients and often lead to lower extremity amputations, which in turn increase the 5-year mortality rate to upwards of 55%. Some studies have shown that just 35% of DFUs heal within a year and that average healing times are longer than 4 months. Neuropathy, poor limb perfusion, infection, epigenetic alterations, aging, and failure to comply with offloading instructions are associated with poor outcomes, but even under the best conditions DFUs still fail to heal at an alarming rate. A particularly frustrating aspect of chronic wound care is that some wounds respond to treatment, while others do not, with no clear reasons for the heterogeneity in patient responsiveness. In general, the mechanisms behind impaired healing are poorly understood.

There is a need to develop methods of evaluating and/or predicting wound healing in patients with chronic wounds such as DFUs. The present invention addresses this need.

### SUMMARY

In some aspects, the present invention is directed to the following non-limiting embodiments:

In some aspects, the present invention is directed to a method of treating, ameliorating, and/or preventing a wound in a subject in need thereof.

In some embodiments, the method comprises:
collecting a first sample from the wound at a first timepoint and determining a first CCL1 (C-C Motif Chemokine Ligand 1) level in the first sample;
administering to the subject a first treatment that promotes wound healing;
collecting a second sample from the wound at a second timepoint after the first timepoint and determining a second CCL1 level in the second sample; and
performing one of the following:
   (a) if the second CCL1 level is higher than the first CCL1 level times by a predetermined value, continuing the administration of the first treatment, or
   (b) if the second CCL1 level is equal to or lower than the CCL1 level times by the predetermined value, administering to the subject a second wound healing treatment that is distinct from the first treatment.

In some embodiments, the wound has shown no significant progress toward healing in 15 days prior to the collection of the first sample.

In some embodiments, the wound is an ulcer.

In some embodiments, the wound is a diabetic ulcer.

In some embodiments, collecting the first sample and/or the second sample comprises swabbing the wound, or debriding the wound and collecting the debrided tissue.

In some embodiments, the first CCL1 level and/or the second CCL2 level is a mRNA level or a protein level of CCL1.

In some embodiments, the first CCL1 level and/or the second CCL2 level is represented as an absolute quantity of CCL1.

In some embodiments, the first CCL1 level and/or the second CCL1 level is represented as a ratio between a quantity of CCL1 and a quantity of a reference biomarker.

In some embodiments, the reference biomarker is TNFAIP6 (tumor necrosis factor, alpha-induced protein 6), APOL1 (apolipoprotein L1), or IRF1 (interferon regulatory factor 1).

In some embodiments, the first timepoint and the second timepoint are separated by about 5 days to about 10 weeks.

In some embodiments, the predetermined value is about 1 or higher.

In some embodiments, the first treatment comprises debriding the wound, applying a compression wrapping, applying a compression stocking, applying dressings promoting a moist environment to the wound, applying a wound offloading device, applying a hyperbaric oxygen therapy, applying an antibiotic, or combinations thereof.

In some embodiments, the second treatment comprises applying a placental membrane derived construct, applying a bioengineered allogeneic cellular construct, or an immunomodulation medication.

In some embodiments, the subject is a mammal.

In some embodiments, the subject is a human.

In some aspects, the present invention is directed to a method of evaluating healing of a wound.

In some embodiments, the method comprises:
collecting a first sample from the wound at a first timepoint and determining a first CCL1 (C-C Motif Chemokine Ligand 1) level in the first sample; and
collecting a second sample from the wound at a second timepoint after the first timepoint and determining a second CCL1 level in the second sample,
wherein
   if the second CCL1 level is higher than the first CCL1 level times by a predetermined value, the healing is significantly progressing, or
   (b) if the second CCL1 level is equal to or lower than the CCL1 level times by the predetermined value, the healing is not significantly progressing.

In some embodiments, the wound has shown no significant progress toward healing in 15 days prior to the collection of the first sample.

In some embodiments, the wound is an ulcer.

In some embodiments, the wound is a diabetic ulcer.

In some embodiments, collecting the first sample and/or the second sample comprises swabbing the wound, or debriding the wound and collecting the debrided tissue.

In some embodiments, the first CCL1 level and/or the second CCL2 level is a mRNA level or a protein level of CCL1.

In some embodiments, the first CCL1 level and/or the second CCL2 level is represented as an absolute quantity of CCL1.

In some embodiments, the first CCL1 level and/or the second CCL1 level is represented as a ratio between a quantity of CCL1 and a quantity of a reference biomarker.

In some embodiments, the reference biomarker is TNFAIP6 (tumor necrosis factor, alpha-induced protein 6), APOL1 (apolipoprotein L1), or IRF1 (interferon regulatory factor 1).

In some embodiments, the first timepoint and the second timepoint are separated by about 5 days to about 10 weeks.

In some embodiments, the predetermined value is about 1 or higher.

In some embodiments, the subject is administered with a treatment for the wound between the collection of the first sample and the collection of the second sample.

In some embodiments, the treatment comprises debriding the wound, applying a compression wrapping, applying a compression stocking, applying dressings promoting a moist environment to the wound, applying a wound offloading device, applying a hyperbaric oxygen therapy, applying an antibiotic, or combinations thereof.

In some embodiments, the method allows for evaluating efficacy of the treatment.

In some embodiments, the subject is a mammal.

In some embodiments, the subject is a human.

### BRIEF DESCRIPTION OF THE DRAWINGS

The following detailed description of exemplary embodiments will be better understood when read in conjunction with the appended drawings. For the purpose of illustrating, non-limiting embodiments are shown in the drawings. It should be understood, however, that the instant specification is not limited to the precise arrangements and instrumentalities of the embodiments shown in the drawings.

FIG. 1 illustrates certain aspects of the experimental design, in accordance with some embodiments. For some subjects, two DFU tissue samples were collected at week 0; one was used for microbial analysis via 16s sequencing, and one was used for NanoString analysis of human gene expression. For some subjects, paired samples were collected after 3-4 weeks as a second time point for NanoString analysis.

FIGS. 2A-2G depict certain aspects of gene expressions in the healing subjects (H) and non-healing subjects (NH), in accordance with some embodiments. FIG. 2A: Hierarchical clustering of DEGs. FIG. 2B: Significantly differentially expressed genes at week 0, Welch's t-test and log FC>|1.5|. FIG. 2C: Significantly differentially expressed genes at week 4, Welch's t-test and log FC>|1.5|. FIG. 2D: Significant difference in fold change of expression week 4 vs. week 0, Welch's t-test. FIG. 2E: Changes over time in paired samples in ssGSEA scores for week 4 vs. week 0 of M1-up and M2a-up gene sets, and in the ratio of M1 ssGSEA score to M2a ssGSEA score over time; student's t-tests. FIG. 2F: M1 macrophage-specific genes at week 0, Welch's t-tests. FIG. 2G: M2a macrophage specific genes at week 0, Welch's t-tests; ** p<0.01, * p<0.05.

FIGS. 3A-3G depict certain aspects of the analysis of the gene expression in accordance with some embodiments. FIG. 3A: Volcano plot; log FC and p value of gene-wise negative binomial generalized linear model (glm). FIG. 3B: Genes identified as significant by glm with p<0.05 and log FC>|1.5| and verified with Welch's t-tests applied to normalized counts; FIGS. 3C-3D: Significant difference in fold change of expression week 4 vs. week 0 and expression over time; Welch's t-test. FIG. 3E: Simple linear regression of gene expression versus number of weeks to healing where slope was significantly non-zero at p<0.05. FIG. 3F: Changes in expression from week 0 to week 4 per patient; green line represents average expression. * p<0.05, ** p<0.01, *** p<0.005.; * p<0.05. ** p<0.01. FIG. 3G: Chart showing how many subjects showed decreasing expression of the three genes shown in FIG. 3F over time.

FIGS. 4A-4D illustrate certain aspects of the relationships between human gene expression and microbial abundance, in accordance with some embodiments. FIG. 4A: Genera detected with relative abundance >0.5%. FIG. 4B: Genes with significant Pearson's correlation coefficient where r>|0.9| and p value <0.05 after Benjamini-Hochberg correction, FIG. 4C: Number of significantly positively and negatively (FIG. 4D) correlated genes by gene set for each species or diversity measure.

FIG. 5 is a heatmap of DEGs week 0 in accordance with some embodiments.

FIG. 6A depicts ssGSEA for raw data at enrollment, in accordance with some embodiments. FIG. 6B depicts ssGSEA for week 4 data, in accordance with some embodiments. Student's t-tests, * p<0.05.

FIG. 7 depicts positive and negative control normalized counts of non-significant macrophage specific genes at week 4, in accordance with some embodiments. Welch's t-tests, * p<0.05.

FIGS. 8A-8B depicts the macrophage specific genes after UQ normalization at enrollment (FIG. 8A) and week 4(FIG. 8B) in accordance with some embodiments. Welch's t-test, * p<0.05.

FIGS. 9A-9H depict CCL1 levels for healing patients and non-healing patients, in accordance with some embodiments.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

The study described herein (“the present study”), using diabetic foot ulcer (DFU) as a non-limiting model for chronic wound, discovered that the expression levels of CCL1 (C-C Motif Chemokine Ligand 1) in wound tissues are an accurate and reliable predicting factor for future progress of wound healing. Specifically, the present study discovered that, in human DFU patients, high CCL1 levels correlate well with healing wounds and low CCL1 levels correlate non-healing wounds. This reliable prediction of wound healing allows the evaluation of chronic wound and the current treatment, as well as provides useful information for selecting future treatments to the wounds.

Accordingly, in some aspects, the present invention is directed to a method of treating, ameliorating and/or preventing a wound in a subject.

In some aspects, the present invention is directed to a method of evaluating wound healing in a subject, or a method of evaluating treatment for wound in a subject.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, peptide chemistry, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

“About” as used herein when referring to a measurable value such as an amount, a temporal duration, and the like, is meant to encompass variations of ±20% or ±10%, in certain embodiments ±5%, in certain embodiments ±1%, in certain embodiments ±0.1% from the specified value, as such variations are appropriate to perform the disclosed methods.

A “disease” is a state of health of an animal wherein the animal cannot maintain homeostasis, and wherein if the disease is not ameliorated then the animal's health continues to deteriorate.

A “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

In one aspect, the terms “co-administered” and “co-administration” as relating to a subject refer to administering to the subject a compound and/or composition of the disclosure along with a compound and/or composition that may also treat or prevent a disease or disorder contemplated herein. In certain embodiments, the co-administered compounds and/or compositions are administered separately, or in any kind of combination as part of a single therapeutic approach. The co-administered compound and/or composition may be formulated in any kind of combinations as mixtures of solids and liquids under a variety of solid, gel, and liquid formulations, and as a solution.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the terms “subject” and “individual” and “patient” can be used interchangeably and may refer to a human or non-human mammal or a bird. Non-human mammals include, for example, livestock and pets, such as ovine, bovine, porcine, canine, feline and murine mammals. In certain embodiments, the subject is human.

As used herein, the term “treatment” or “treating” is defined as the application or administration of a therapeutic agent, i.e., a compound useful within the disclosure (alone or in combination with another pharmaceutical agent), to a patient, or application or administration of a therapeutic agent to an isolated tissue or cell line from a patient (e.g., for diagnosis or ex vivo applications), who has a disease or disorder and/or a symptom of a disease or disorder, with the purpose to cure, heal, alleviate, relieve, alter, remedy, ameliorate, improve or affect the disease or disorder and/or the symptoms of the disease or disorder. Such treatments may be specifically tailored or modified, based on knowledge obtained from the field of pharmacogenomics.

Methods of Treating, Ameliorating, and/or Preventing Wounds

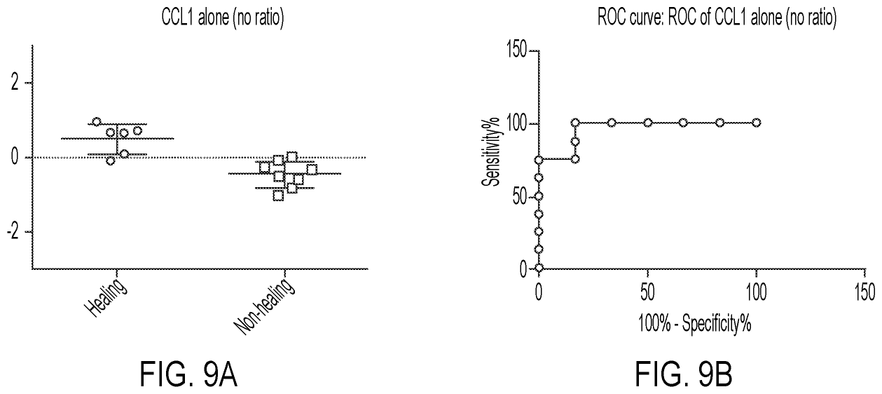

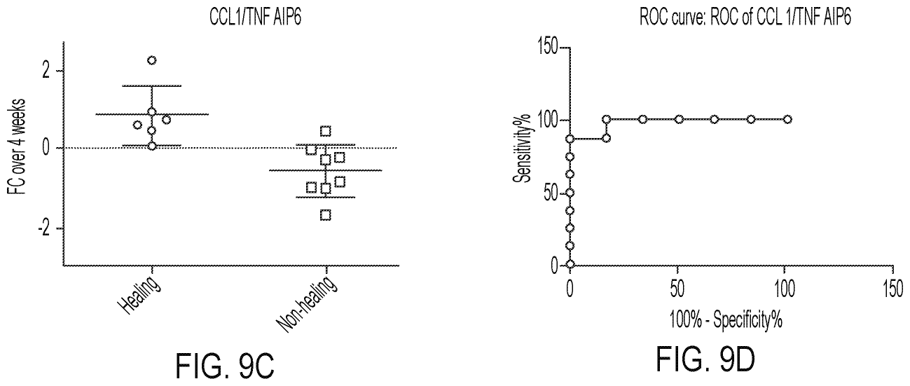

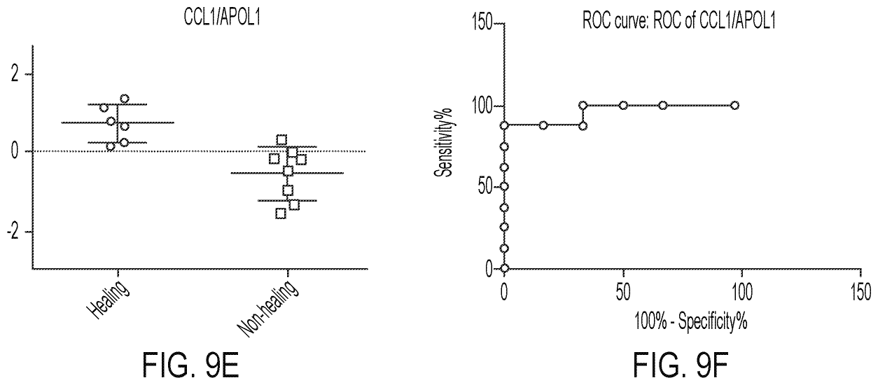

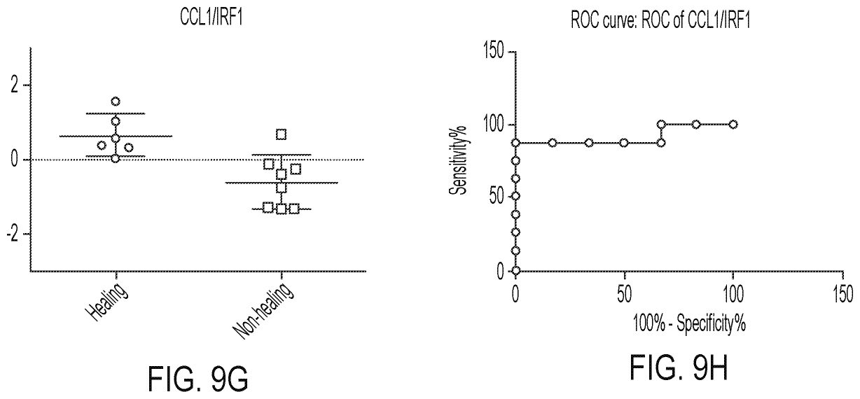

Using diabetic foot ulcer as a non-limiting example, the present study discovered that CCL1 levels in wound tissues can provide accurate and reliable evaluation to the wound healing (see e.g., FIGS. 9A-9B), thereby providing guidance to future treatments of the wound.

Accordingly, in some aspects, the instant specification is directed to a method of treating, ameliorating and/or preventing wound in a subject in need thereof.

In some embodiments, the method include:
collecting a first sample from the wound at a first timepoint and determining a first CCL1 (C-C Motif Chemokine Ligand 1) level in the first sample;
administering to the subject a first treatment that promotes wound healing;
collecting a second sample from the wound at a second timepoint after the first timepoint and determining a second CCL1 level in the second sample; and
performing one of the following:
   (a) if the second CCL1 level is higher than the first CCL1 level times by a predetermined value, continuing the administration of the first treatment or discontinuing the first treatment, or
   (b) if the second CCL1 level is equal to or lower than the CCL1 level times by the predetermined value, administering to the subject a second wound healing treatment that is distinct from the first treatment.

In some embodiments, the wound is a chronic wound. In some embodiments, the wound has shown no significant progress toward healing (such as failed to achieve sufficient healing) in about 7 days, such as about 10 days, about 2 weeks, about 15 days, about 20 days, about 3 weeks, about 4 weeks, or about 30 days. In some embodiments, the wound has shown no significant progress toward healing after standard care for the time period set forth above.

In some embodiments, the wound is a nonhealing wound; an infected wound such as an infected surgical wound or an infected traumatic wound; or an ulcer such as a diabetic ulcer (e.g., a diabetic foot ulcer), an arterial ulcer, a venous ulcer, a pressure ulcer, an ischemic ulcer, and the like.

In some embodiments, collecting the first sample and/or the second sample comprises swabbing the wound. In some embodiments, collecting the first sample and/or the second sample comprises debriding the wound and collecting the debrided tissue. Debridement is the medical removal of dead, damaged, or infected tissue of or associated with wounds. The removed tissues are used as samples according to the method herein in some embodiments. In some embodiments, the debridement per se is also considered an example of the first treatment that promotes wound healing.

In some embodiments, the first CCL1 level and/or the second CCL2 level is a mRNA level or a protein level of CCL1. Methods of quantifying CCL1 mRNA or protein (or mRNAs or proteins of any genes) are well known in the art. Non-limiting examples of mRNA quantification methods include RNA based methods such as RT-PCR, northern blotting, and the like. None limiting examples of protein quantification methods include enzyme-linked immunoassay (ELISA), western blotting, and the like.

In some embodiments, the first CCL1 level and/or the second CCL2 level is represented as an absolute quantity of CCL1. In some embodiments, the absolute quantity of CCL1 is determined as a concentration of the CCL1, which can be estimated using, for example, total volume, total weight, total protein level, total RNA level, total DNA level, or total nucleic acid level as a reference.

In some embodiments, the first CCL1 level and/or the second CCL1 level is represented as a ratio between a quantity of CCL1 and a quantity of a reference biomarker. In some embodiments, the reference biomarker is an mRNA or a protein.

In some embodiments, the reference biomarker is TNFAIP6 (tumor necrosis factor, alpha-induced protein 6), APOL1 (apolipoprotein L1), or IRF1 (interferon regulatory factor 1). Referring to FIGS. 9C-9H, the present study discovered that the ratios of CCL1 to any of these three pro-inflammatory genes were highly accurate at predicting wound healing.

In some embodiments, the first timepoint and the second timepoint are separated by about 5 days to about 10 weeks, such as by about 1 week to about 8 weeks, about 2 weeks to about 6 weeks, or about 3 weeks to about 5 weeks. In the first timepoint and the second timepoint are separated by about 5 days, about 1 week, about 10 days, about 2 weeks, about 14 days, about 3 weeks, about 4 weeks, about 30 days, about 5 weeks, about 6 weeks, about 8 weeks, or about 10 weeks.

In some embodiments, the predetermined value (the first CCL1 level is compared to the product of second CCL1 level and the predetermined value) is about 1 or higher. In some embodiments, the predetermined value is about 0.9, about 0.95, about 1, about 1.05, about 1.1, about 1.2, about 1.25, about 1.4, or about 1.5.

In some embodiments, the first treatment includes debriding the wound, applying a compression wrapping, applying a compression stocking, applying dressings promoting a moist environment to the wound, applying a wound offloading device, applying a hyperbaric oxygen therapy, applying an antibiotic, or combinations thereof. In some embodiments, the first treatment includes applying a placental membrane derived construct, applying a bioengineered allogeneic cellular construct, an immunomodulation medication, or combinations thereof.

In some embodiments, the second treatment is considered more aggressive than the first treatment and is used when the first treatment, as evaluated according to the CCL1, did not significantly promote the healing of the wound. In some embodiments, the second treatment includes debriding the wound, applying a compression wrapping, applying a compression stocking, applying dressings promoting a moist environment to the wound, applying a wound offloading device, applying a hyperbaric oxygen therapy, applying an antibiotic, or combinations thereof. In some embodiments, the second treatment includes applying a placental membrane derived construct, applying a bioengineered allogeneic cellular construct, or an immunomodulation medication. Examples of immunomodulation medications used to treat wounds include steroids, immunosuppressive medications such as azathioprine or ciclosporin.

In some embodiments, the subject is a mammal, such as a human.

### Method of Evaluating Wound Healing

Using diabetic foot ulcer as a non-limiting example, the present study discovered that CCL1 levels in wound tissues can provide accurate and reliable evaluation to the wound healing (see e.g., FIGS. 9A-9B).

Accordingly, in some aspects, the present invention is directed to a method of evaluating the healing of a wound.

In some embodiments, the method includes:
collecting a first sample from the wound at a first timepoint and determining a first CCL1 (C-C Motif Chemokine Ligand 1) level in the first sample; and
collecting a second sample from the wound at a second timepoint after the first timepoint and determining a second CCL1 level in the second sample,
wherein
   if the second CCL1 level is higher than the first CCL1 level times a predetermined value, the healing is making sufficient progress, and
   if the second CCL1 level is equal to or lower than the CCL1 level times the predetermined value, the healing is not making sufficient progress.

In some embodiments, the wound is a chronic wound. In some embodiments, the wound has shown no significant progress toward healing (such as failed to achieve sufficient healing) in about 7 days, such as about 10 days, about 2 weeks, about 15 days, about 20 days, about 3 weeks, about 4 weeks, or about 30 days. In some embodiments, the wound has shown no significant progress toward healing after standard care for the time period set forth above.

In some embodiments, the wound is a nonhealing wound; an infected wound such as an infected surgical wound or an infected traumatic wound; or an ulcer such as a diabetic ulcer (e.g., a diabetic foot ulcer), an arterial ulcer, a venous ulcer, a pressure ulcer, an ischemic ulcer, and the like.

In some embodiments, collecting the first sample and/or the second sample comprises debriding the wound and collecting the debrided tissue.

In some embodiments, the first CCL1 level and/or the second CCL2 level is a mRNA level or a protein level of CCL1.

In some embodiments, the first CCL1 level and/or the second CCL2 level is represented as an absolute quantity of CCL1.

In some embodiments, the first CCL1 level and/or the second CCL1 level is represented as a ratio between a quantity of CCL1 and a quantity of a reference biomarker. In some embodiments, the reference biomarker is an mRNA, or a protein.

In some embodiments, the reference biomarker is TNFAIP6 (tumor necrosis factor, alpha-induced protein 6), APOL1 (apolipoprotein L1), and/or IRF1 (interferon regulatory factor 1).

In some embodiments, the first timepoint and the second timepoint are separated by about 5 days to about 10 weeks, such as by about 1 week to about 8 weeks, about 2 weeks to about 6 weeks, or about 3 weeks to about 5 weeks. In the first timepoint and the second timepoint are separated by about 5 days, about 1 week, about 10 days, about 2 weeks, about 14 days, about 3 weeks, about 4 weeks, about 30 days, about 5 weeks, about 6 weeks, about 8 weeks, or about 10 weeks.

In some embodiments, the predetermined value (the first CCL1 level is compared to the product of second CCL1 level and the predetermined value) is about 1 or higher. In some embodiments, the predetermined value is about 0.9, about 0.95, about 1, about 1.05, about 1.1, about 1.2, about 1.25, about 1.4, or about 1.5.

In some embodiments, between the collections of the first sample and the second sample, the subject is administered with a treatment for the wound.

In some embodiments, the treatment includes debriding the wound, applying a compression wrapping, applying a compression stocking, applying dressings promoting a moist environment to the wound, applying a wound offloading device, applying a hyperbaric oxygen therapy, applying an antibiotic, or combinations thereof.

In some embodiments, the method of evaluating the healing of the wound is also a method of evaluating the treatment for the wound.

In some embodiments, the subject is a mammal, such as a human.

### EXAMPLES

The instant specification further describes in detail by reference to the following experimental examples. These examples are provided for purposes of illustration only, and are not intended to be limiting unless so specified. Thus, the instant specification should in no way be construed as being limited to the following examples, but rather, should be construed to encompass any and all variations which become evident as a result of the teaching provided herein.

### Example 1

Diabetic foot ulcers (DFUs) are a common occurrence for diabetic patients and are notoriously difficult to treat because the mechanisms behind why some patients heal and others do not are poorly understood. However, chronic inflammation and the failure of macrophages to transition between phenotypes have been implicated.

The study described herein (“the present study”) analyzed a panel of 227 inflammation-related genes (including markers of multiple macrophage phenotypes such as M1, M2a, and M2c) in human healing vs. non-healing DFUs from 27 subjects. Paired samples from a subset of subjects were analyzed for changes over time and for the relationships to composition of the microbiome in the wound environment, determined using 16s sequencing. Many genes were expressed at significantly higher levels in non-healing DFUs compared to healing DFUs, suggesting increased inflammation and/or numbers of immune cells. However, a substantial number of non-healing DFUs exhibited lower inflammation-related gene expression compared to healing DFUs, suggesting that heterogeneity in wound microenvironment among patients may at least partially explain the observed variability in the response to treatment. When the data were normalized to account for these differences, healing and non-healing DFUs still differed in how expression of the genes changed over time and how they correlated with microbial abundance. For example, healing and non-healing DFUs showed opposite trends in expression of TNAIP6 and RPL37A over time. Time course analysis of healing DFUs revealed that as the wounds approached full closure, expression of GXYLT2, IL10, and TNIP3 decreased, whereas no clear patterns were observed in non-healing DFUs. In non-healing DFUs, many genes were correlated with microbial diversity and with particular species such as S. epidermidis and S. aureus, but these trends were not observed in healing DFUs, suggesting less colonization with microbes and/or less communication with human immune cells. Overall, the results indicate that sustained inflammation over time contributes to poor healing outcome and the microbiome is a critical regulator of immune cell behavior in non-healing DFUs, although the present study also found substantial heterogeneity that can be important for differences in patient responsiveness to treatment.

### Example 2

Diabetic foot ulcers (DFUs) is a major complication for diabetic patients. They occur in approximately 15% of patients and often lead to lower extremity amputations, which in turn increase the 5-year mortality rate to upwards of 55%. Some studies have shown that just 35% of DFUs heal within a year and that average healing times are longer than 4 months. Neuropathy, poor limb perfusion, infection, epigenetic alterations, aging, and failure to comply with offloading instructions are associated with poor outcomes, but even under the best conditions, DFUs still fail to heal at an alarming rate. A particularly frustrating aspect of chronic wound care is that some wounds respond to treatment, while others do not, with no clear reasons for the heterogeneity in patient responsiveness. In general, the mechanisms behind impaired healing are poorly understood, but dysfunctional immune cell activation and recruitment have been implicated. While investigations into immune cell behavior using animal models have been instrumental to advancement of mechanistic understanding, diabetic animal models are inadequate for the study of chronic wounds, especially because they fail to replicate the heterogeneous nature of the human response to treatment in which some wounds never heal. There is a need for human clinical research to increase understanding of maladaptive immune cell function in healing and non-healing DFUs to better understand this heterogeneity so that more targeted therapeutics can be developed.

In the absence of disease, wound healing is a dynamic process that occurs in four phases, each of which is regulated by macrophages with distinct phenotypes. In order for healing to occur, macrophages must transition from a pro-inflammatory (M1) to a pro-healing (M2) phenotype, although the extent of diversity of the M2 population in particular is not known. Several M2 subtypes have been described, especially those that form in response to interleukin-4 (IL4) (M2a) and IL10 (M2c) in vitro, although it is hotly debated how well these phenotypes represent those that are found in vivo. Nonetheless, studies using diabetic animal models have reported that failure of macrophages to transition from M1 to M2 is associated with impaired wound healing. The reasons why macrophages fail to make this transition may include impaired efferocytosis, hyperglycemia, hypoxia, chronic infection, and likely many others. Communication with the microbiome has been shown to be regulate macrophage phenotype in animal studies and in vitro studies, but has not yet been linked to immune cell behavior in human chronic wounds.

Changes in immune-related processes in human chronic wounds have been investigated using gene expression analyses of whole wound tissue because this technique is amenable to clinical sample collection, since samples can be easily collected into non-toxic and non-noxious buffers with minimal added time or effort. For example, RNAseq has been used to compare gene expression signatures in non-healing DFUs from 11 subjects to acute wound tissue from healthy subjects. They reported a pattern of gene expression that suggested decreased neutrophil and macrophage recruitment. Other studies have used gene expression profiling to compare healing and non-healing DFUs. The ratio of four M1 markers to three M2 markers in debrided wound tissue decreased over time for healing DFUs but not non-healing DFUs, although the genes were not specific to macrophages so conclusions about macrophage phenotype could not be drawn. A small panel of macrophage-specific genes were also analyzed and it was found that non-healing DFUs expressed relatively higher levels of M1 markers than healing DFUs. However, this analysis was not conducted over time, which is a consideration for macrophage phenotype. Given that these studies implicated inflammation as mediators of chronic wound healing, one of the goals of the present was to compare changes in inflammation-related gene over time in human healing and non-healing DFUs and to investigate the influence of the microbiome as a potential mediator.

To this end, debrided tissue samples were collected from chronic DFUs from 27 subjects as well as paired samples 4 weeks later for a subset of these subjects (n=14) to analyze changes in human gene expression over time and microbial composition between healing and non-healing DFUs, defined based on whether wound closure was complete by 12 weeks from initial sample collection. The present study chose to use NanoString™ for multiplex gene expression analysis of a custom-curated panel of genes related to macrophage phenotype and crosstalk with microbes, as opposed to whole transcriptome analysis such as RNAseq, to increase the number of patient samples that could be processed and to reduce the risks associated with RNA-seq such as biological and technical noise, inconsistency in reporting methodology, and processing constraints on low-quantity RNA samples. 16s RNA sequencing was used to characterize microbial abundance.

### Example 3: Methods

### Study Design

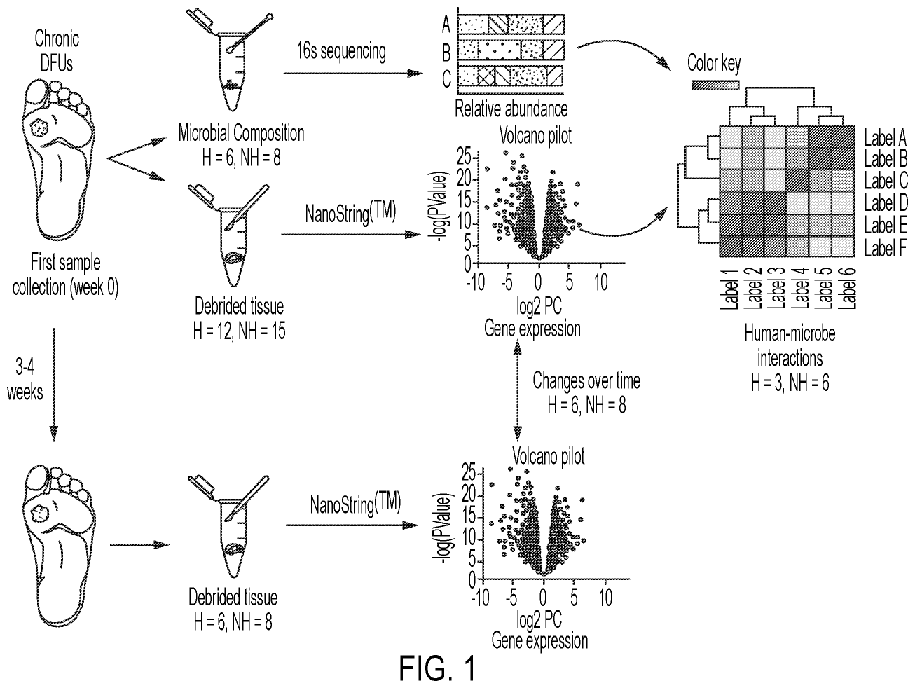

Twenty-nine subjects were recruited over a 2-year period from the Drexel University Wound Healing Center after providing written consent and in compliance with the study protocol approved by the Drexel University Institutional Review Board. Inclusion criteria included being 18+ years of age with a diagnosis of diabetes and an ulcer that had been open for at least 8 weeks at the time of enrollment. Exclusion criteria included insufficient vascular perfusion (ankle brachial index <0.75) and those who presented with signs or symptoms of invasive or systemic infection such as cellulitis or abscess. Subjects were treated according to standard wound care procedures determined by the physician, including weekly or biweekly debridement with a sharp scalpel, offloading, topical antibiotics as needed, and moist wound dressings. Debrided DFU tissue samples were collected at every visit until complete wound closure, amputation, death, or until the study ended (no earlier than 20 weeks since enrollment of the last subject). Debrided tissue samples were immediately collected into vials of RNAlater as described below. The status of the wound was followed at subsequent clinical visits and samples were classified as healing or non-healing based on whether the wound was fully healed at 12 weeks from sample collection. Additionally, two subjects healed within 16-25 weeks from the initial sample collection, so the first samples collected from these subjects were counted as non-healing and then the sample collected exactly 12 weeks prior to healing was counted as a healing sample. Some of the samples were used in preliminary studies for optimization of RNA extraction, and some samples could not be analyzed due to insufficient RNA or loss of subjects to follow-up, resulting in n=12 healing DFUs and n=15 non-healing DFUs at week 0, and paired samples from n=6 healing DFUs and n=8 non-healing DFUs at the second time point 3-4 weeks later (Table 1, FIG. 1). Similarly, n=3 healing DFUs and n=6 non-healing DFUs had paired samples for microbial analysis and human gene expression.

TABLE 1
Subject characteristics.

| | Healing (n = 12) | Non-healing (n = 15) |
|---|---|---|
| Average Age | 66.7 ± 13.25 | 62.2 ± 5.75 |
| Average BMI | 33.7 ± 5.04 | 36.8 ± 7.52 |
| % Female | 33.3 | 26.7 |
| % Current smoker | 33.3 | 40 |

### Debrided Tissue Sample Collection

Two types of samples were collected from patients. First an initial swab of the wound was placed into RNAlater for microbial analysis. Next, a deeper debridement was conducted, in accordance with standard wound care guidelines, and this tissue was also collected into a second vial of RNALater for human gene expression analysis. Collected tissue were immediately placed in RNAlater (Ambion, Carlsbad, CA) and stored at 4° C. overnight. They were then transferred to −80° C. until processing.

### 16s Sequencing and Analysis of Microbial Composition

### RNA Extraction and NanoString

Tissue samples were thawed at room temperature, RNAlater was removed, and total RNA was isolated using Trizol followed by purification with Qiagen RNeasy (Qiagen, Inc., CA, USA) according to manufacturer's protocol, as described in Nassiri et al. (The Journal of investigative dermatology 135, 1700-1703 (2015)). DNA was inactivated with DNAse I Amplification Grade (Invitrogen, Carsbad, CA, USA). NanoString gene expression analysis was run according to the manufacturer's recommended protocol using 100 ng per sample and a custom code set of 227 genes that were selected from the literature as being differentially regulated according to macrophage phenotypes, host-microbe communication, and others related to wound healing (Table 2). For macrophage phenotype markers, genes were grouped based on whether they were found to be upregulated or downregulated with M1, M2a, or M2c polarization compared to unactivated controls (M0) using in vitro studies with defined chemical stimuli (lipopolysaccharide and interferon-gamma for M1, interleukin (IL)-4 and IL-13 for M2a, and IL10 for M2c).

TABLE 2
Genes and gene sets assessed using NanoString.

| Angiogenesis | Bacteria | M1-up | M1-down | M2a-up | M2a-down |
|---|---|---|---|---|---|
| ANGPT1 | C3ar1 | ADORA2A | ANKRD22 | ABCG2 | ALDH1A2 |
| ANGPT2 | C5AR1 | APOBEC3A | PRSS8 | ALOX15 | ATOH8 |
| CCL18 | CAMP | APOL1 | CFH | CACNA1G | CABLES1 |
| COL1A1 | CFD | ASPHD2 | NEURL3 | CACNB4 | CCDC85C |
| COL4A1 | DEFB1 | C1orf61 | FBXO2 | CCL22 | CCL28 |
| CTGF | DEFB3 | CASP1 | SERPING1 | CCL26 | CDH1 |
| FGF2 | DEFB4 | CCL1 | KRT7 | CD1C | CHDH |
| NOTCH1 | LYZ | CCL19 | | CH25H | EHF |
| PECAM1 | MYD88 | CCL5 | | COL5A3 | ENHO |
| PF4 | OR2AT4 | CCL8 | | CR2 | FABP4 |
| SPP1 | REG3G | CCR7 | | CRB2 | IL17RB |
| TEK | S100a8 | CD38 | | DACT1 | MAOA |
| TGFB1 | S100a9 | CD80 | | DNASE1L3 | MUCL1 |
| TIE1 | SLPI | CFB | | DUOX1 | NIPAL1 |
| VEGFA | TLR1 | CLEC4D | | DUOXA1 | PLEKHA6 |
| | TLR2 | CLEC4E | | FAM110B | RAMP1 |
| | TLR3 | CMPK2 | | FCGR2B | S100A1 |
| | TLR4 | CRISPLD2 | | FOXQ1 | SEMA3G |
| | TLR5 | CSF3 | | GCNT3 | ST8SIA6 |
| | TLR6 | CXCl10 | | IL21R | SYT17 |
| | TLR7 | CXCL11 | | LIMA1 | TSPAN7 |
| | TLR8 | CXCL9 | | LRRC4 | |
| | TLR9 | EBI3 | | MEST | |
| | | EPHA2 | | MORC4 | |
| | | GBP1 | | MRC1 | |
| | | GBP4 | | MS4A6E | |
| | | GBP5 | | NEK10a | |
| | | GCH1 | | NEK10b | |
| | | HAPLN3 | | OLFML3 | |
| | | HCAR3 | | PALD1 | |
| | | HLA-DOA | | PCSK1 | |
| | | HLA-DOB | | PDGFB | |
| | | HSH2D | | SIGLEC12 | |
| | | IDO1 | | SLC25A48 | |
| | | IDO2 | | SNAI3 | |
| | | IFI44L | | TAL1 | |
| | | IFITM1 | | TGM2 | |
| | | IFITM3 | | TIMP3 | |
| | | IL15 | | TNFRSF11A | |
| | | IL15RA | | VTN | |
| | | IL1b | | WDR66 | |
| | | IL27 | | CLEC4G | |
| | | IL32 | | PLCB1 | |
| | | IL3RA | | TNFRSF1A | |
| | | IL6 | | TNFRSF1B | |
| | | IL8 | | | |
| | | IRF1 | | | |
| | | ISG15 | | | |
| | | ISG20 | | | |
| | | ITK | | | |
| | | LAG3 | | | |
| | | MN1 | | | |
| | | MT1M | | | |
| | | NCF1 | | | |
| | | NCF1B | | | |
| | | NNMT | | | |
| | | NOD2 | | | |
| | | OASL | | | |
| | | PDE4B | | | |
| | | PTGES | | | |
| | | PTGS2 | | | |
| | | RIMS2 | | | |
| | | RSAD2 | | | |
| | | TNFAIP6 | | | |
| | | TNIP3 | | | |
| | | UBD | | | |
| | | VEGFA | | | |
| | | XAF1 | | | |
| | | ZBP1 | | | |
| | | SERPINB7 | | | |
| | | IGFBP4 | | | |

| | M2c- | Wound | | | |
|---|---|---|---|---|---|
| M2c-up | down | healing | M1 specific | M2a specific | Housekeeping |
| TIMP1 | PCOLCE2 | ABCC8 | APOL1 | ALOX15 | EIF2B2 |
| SERPINA1 | F5 | CASP1 | ASPHD2 | CCL26 | GAPDH |
| CD163 | | CASP12 | CCL19 | CLEC4G | PPIA |
| FPR1 | | CCNA1 | CLEC4D | PLCB1 | RPL37A |
| LIN7A | | KDM5A | NOD2 | RAMP1 | TRFC |
| PLOD2 | | KDM6B | XAF1 | WNT5B | UBE2D2 |
| KCNJ15 | | NLRP3 | | | VCP |
| SH3PXD2B | | NUR77 | | | VPS29 |
| VCAN | | PI9 | | | |
| MCTP2 | | SETD7 | | | |
| GXYLT2 | | | | | |
| IL10 | | | | | |
| CD300E | | | | | |
| MERTK | | | | | |

### NanoString Data Processing

Raw counts from NanoString were normalized to internal positive and negative controls according to the manufacturer's recommendations. First, positive control normalization was performed by multiplying endogenous counts by their sample specific scaling factor, calculated using the geometric mean of all 6 positive controls. Next, the background threshold method was used to account for noise. The average of the 8 negative controls was subtracted from all endogenous counts on per-sample basis. These steps were performed for enrollment and week 4 data sets separately. Samples were excluded from analysis if more than 5000 of genes were not expressed above background levels.

Significantly differentially expressed genes (DEGs) were identified between healing (H) and non-healing (Nil) groups using Welch's t-test and p-values <0.05 and log 2 fold change greater than 1.5. DEGs were plotted as heatmaps using the ComiplexHeatmap package in R and bar plots were created in GraphPad Prism. For macrophage-specific analyses, the 12 genes were plotted individually. Lastly, gene set enrichment scores were calculated for all gene sets (Table 2) using the ssGSEA function of the GSVA package. Student's t-test was used to determine significance between groups at a p-value of 0.05. To evaluate changes over time, the fold change values of individual genes or ssGSEA scores at week 4 compared to week 0 were calculated as well as the ratio of these changes. Student's t-tests were used to establish significance between healing and non-healing DFU samples. P values were not adjusted for multiple comparisons because a small number of genes was investigated and a log 2 FC of at least 1.5 was used for significance.

### Upper Quartile Normalization for Analyses of Cellular Behavior

To account for differences in the quantity of immune cells between groups, data were then normalized to the upper quartile (UQ) using the EDASeq package in R. This method is typically applied to bulk RNA-seq data, but has been shown effective for reducing unwanted variation within NanoString data (Bhattacharya et al. Briefings in bioinformatics 22, bbaa163, doi:10.1093/bib/bbaa163 (2021)). The edgeR library was used for gene-wise negative binomial generalized linear modeling (glm) to estimate statistical significance between groups. Log 2 fold change values of healing vs. non-healing DFUs and −log 10 p-values were used to create a volcano plot with ggplot2. Genes were identified as statistically significant between groups if both the absolute value of log 2 fold change was greater than 1.5 and the p-value was less than 0.05. Significant DEGs were visualized with a heatmap using the ComplexHeatmap package. Identified genes were plotted in Prism using the normalized counts, not fitted, and confirmed to be statistically significant between groups using Welch's t-test and a significance level of p<0.05. Genes in which more than 50% of samples had no expression above negative controls were excluded. Fold change values of week 4 compared to week 0 were calculated for each gene for the subset of subjects with matched samples at both 0 week and 4 week time points. Welch's t-tests and criteria for significance were performed as previously described.

### Linear Regression Analysis

Linear regression analyses were performed for gene expression in the healing DFU samples as a function of number of weeks remaining until complete wound closure. The lm function within the stats package was used to calculate p-value and F-statistic. P-values were adjusted using the Benjamini-Hochberg (BH) correction. Genes with an adjusted p-value <0.05 and a ratio of F-statistic to number of samples greater then 0.5 were considered significant. Counts were then plotted in GraphPad Prism and the simple linear regression function was used to identify which genes had significantly non-zero slopes. Finally, these genes were analyzed on a per-subject level using matched samples for each subject using GraphPad Prism.

### Human-Microbe Correlation Analyses

Welch's t-tests were used to determine if there was statistical difference between diversity indexes or abundance of genera between healing and non-healing groups. Pearson's correlations were calculated for each gene to abundance of genera, Shannon index, phylogenetic diversity, and the number of observed species for the healing and non-healing samples separately. P-values were adjusted using the BH correction. Correlations with adjusted p-value <0.05 and |r|>0.9 were considered significant. Heatmaps were created using the ComplexHeatmap, RColorBrewer, and circlize packages in R. The non-healing dataset was clustered row-wise by Euclidean distance and single linkage. Pie charts representing the number of genes from each gene set were created in GraphPad Prism.

### Example 4

### Differentially Expressed Genes Before Normalization

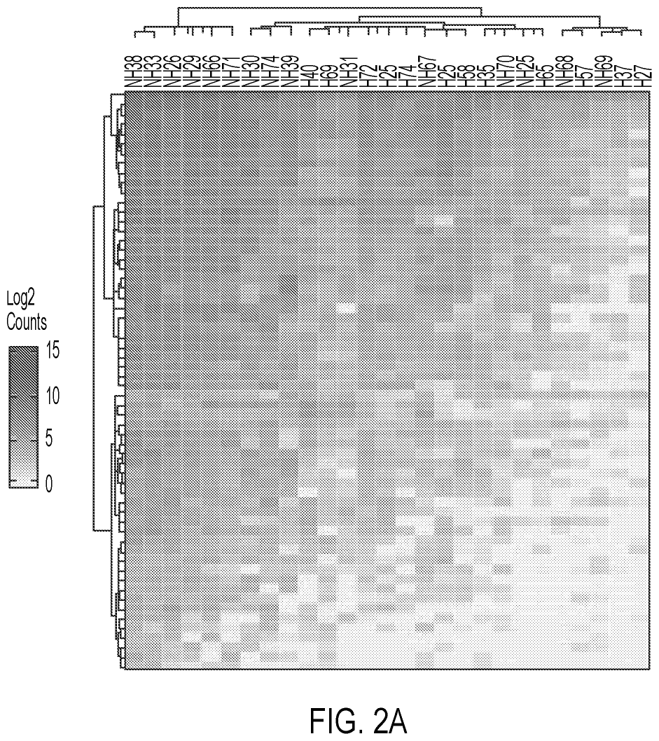

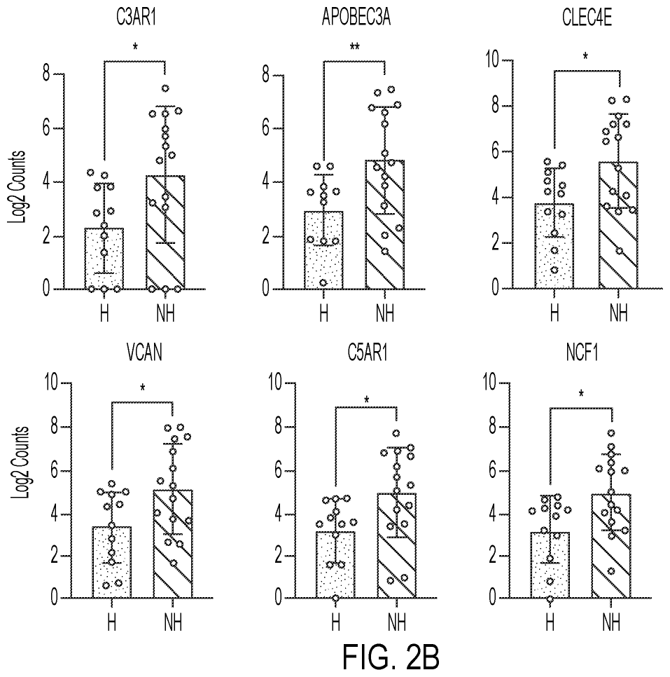

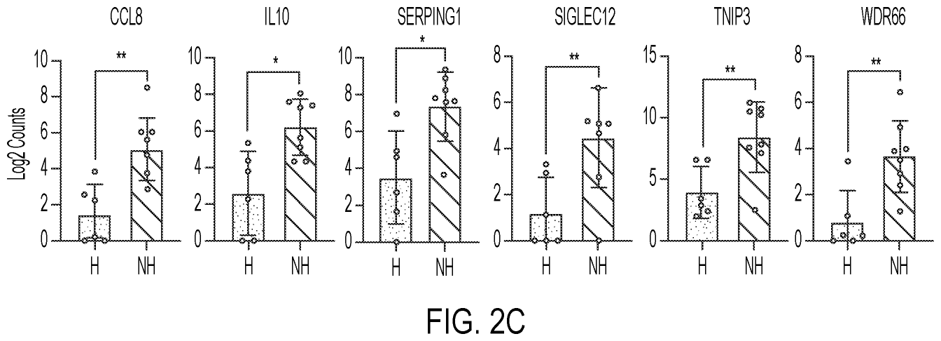

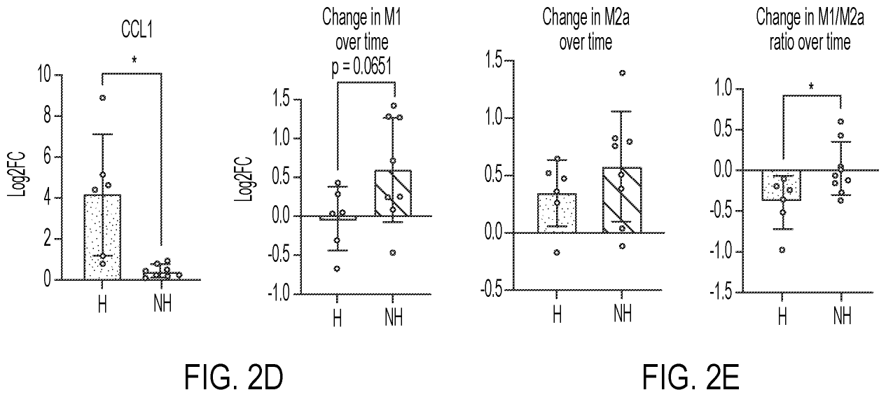

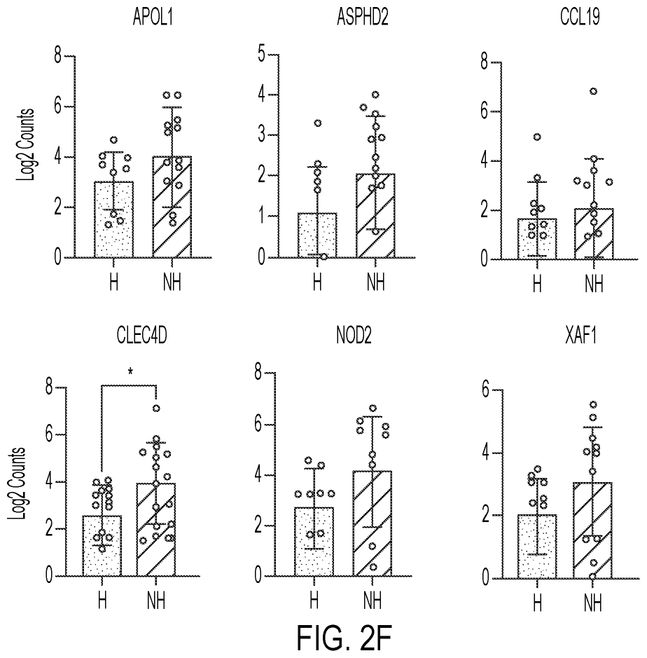

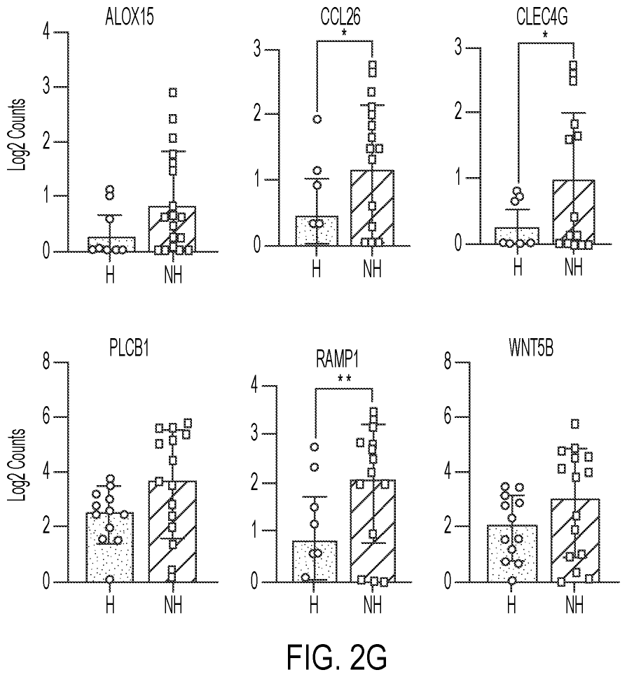

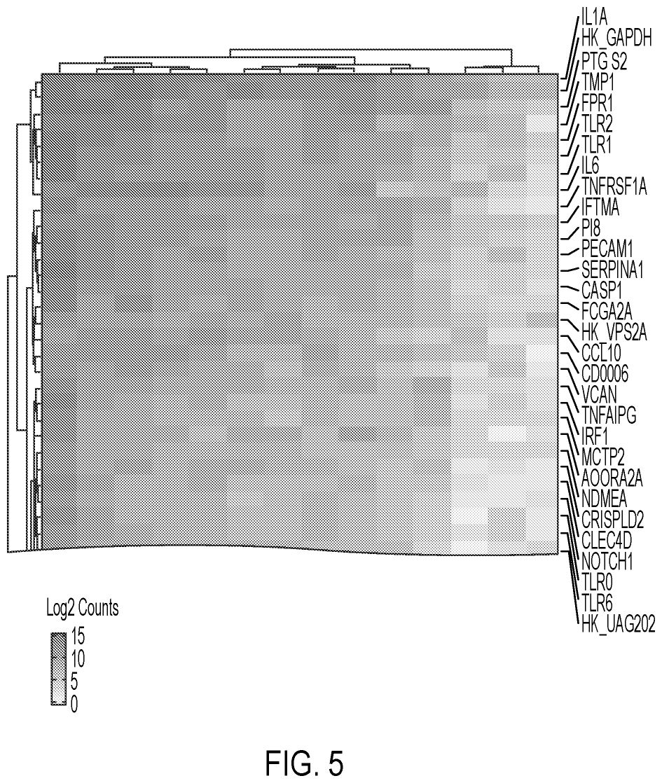

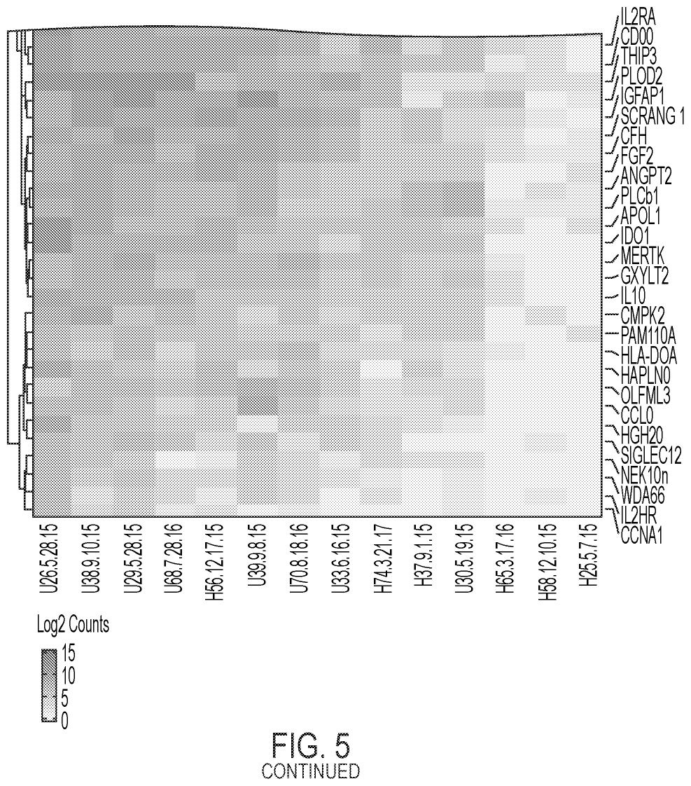

A custom-curated list of 227 genes related to inflammation, macrophage phenotype, crosstalk with microbes, and wound healing more generally were used to characterize differences between DFUs that ultimately healed by 12 weeks (n=12) compared to those that remained open at that time point (n=15). There were 85 significant DEGs between these two groups at the first time point (Table 4) and 57 DEGs at week 4 (Table 5). All were significantly upregulated in non-healing DFUs compared to healing DFUs, with no significantly downregulated genes. Although a majority of the non-healing DFUs (9 of 15) exhibited higher expression of these inflammation-related genes compared to healing DFUs, 6 of the non-healing DFUs exhibited similar or lower levels, clustering together with healing DFUs (FIG. 2A). This pattern was true even at the week 4 time point, when the healing DFUs were all within 8 weeks of healing (FIG. 5).

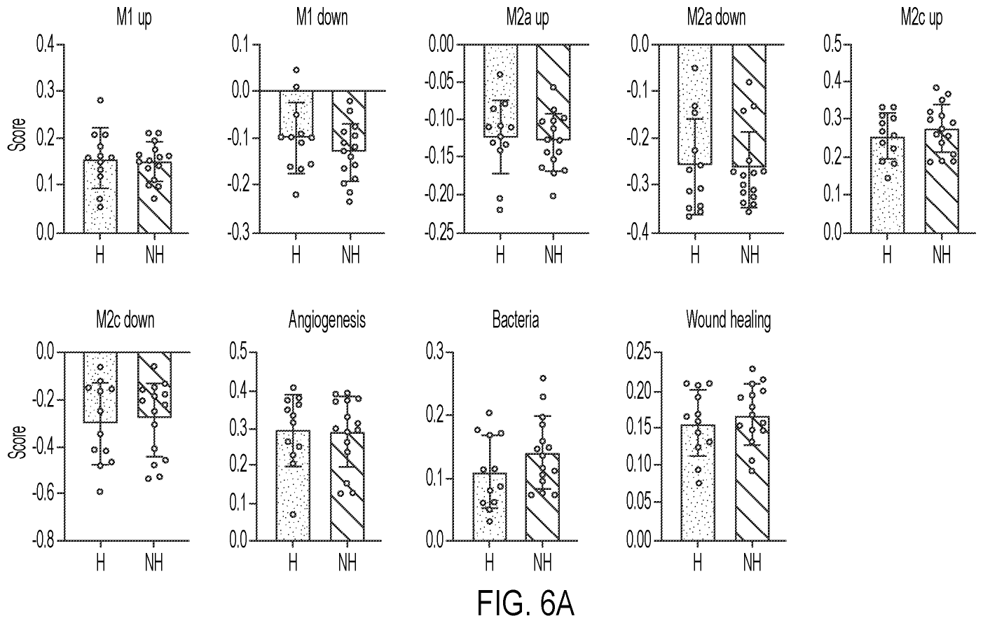

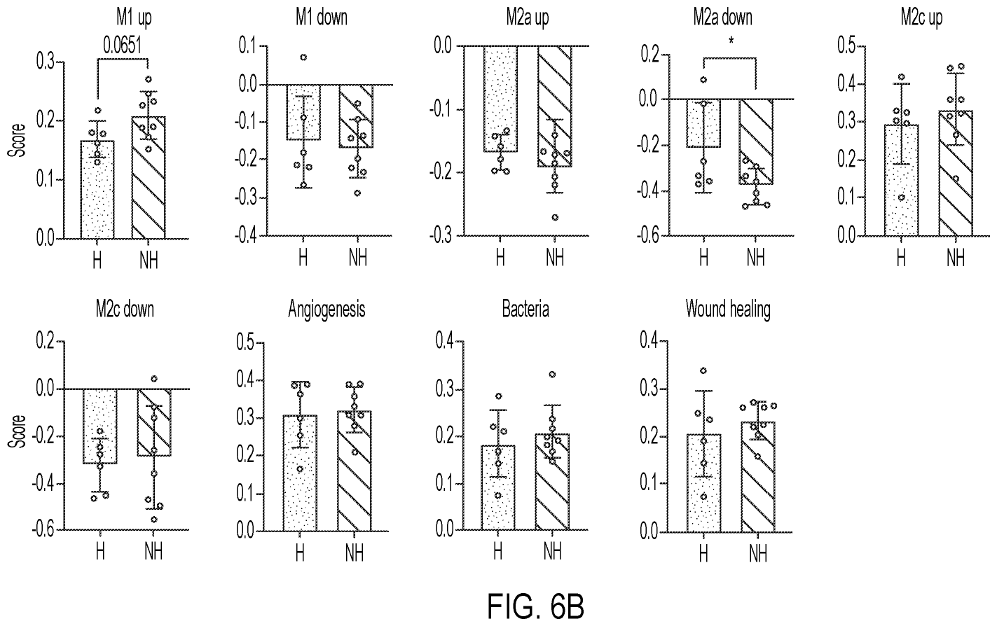

Of the top 6 most differentially expressed genes between the groups (FIG. 2B), 3 were markers associated with the pro-inflammatory M1 phenotype (APOBEC3A, CLEC4E, and NCF1). C3AR1 and C5AR1 are involved with host-microbe communication, and VCAN was included as an M2c macrophage phenotype marker, but is also involved in regulation of cell migration and extracellular matrix (ECM) assembly. At week 4, the top 6 most differentially expressed genes (FIG. 2C) were related to M1 (CCL8, TNIP3), M2a (SIGLEC12, WDR66), and the anti-inflammatory and M2c-promoting cytokine IL10, while SERPING1 is an M1 downregulation marker. 28 genes were upregulated in non-healing DFUs at both time points (Table 3). 9 were M1 markers and 7 were M2c markers. Lastly, how gene expression changed overtime were analyzed in paired samples, and only CCL1 significantly differed between healing and non-healing DFUs in terms of change compared to week 0; CCL1 expression increased in all healing DFUs between week 0 and week 4, whereas it only slightly increased in non-healing DFUs (FIG. 2D). How sets of genes changed over this same time period were also analyzed (FIG. 2E). While none of the analyzed gene sets were significantly differentially enriched between groups at week 0 (FIG. 6A), the gene set associated with downregulation upon M2a macrophage polarization was significantly enriched at week 4 in the healing DFUs compared to non-healing DFUs (FIG. 6B). Enrichment of the M1 macrophage gene set and the ratio between the M1 and M2a gene sets both increased over time to a greater extent in non-healing DFUs compared to healing DFUs (p=0.06 and p=0.05, respectively).

TABLE 3
Genes that were significantly upregulated by non-healers at both time points.

| M1 | M2a | M2c | Angiogenesis | Bacteria | Wound Healing |
|---|---|---|---|---|---|
| APOL1 | IL21R | CD300E | NOTCH1 | TLR2 | CASP1 |
| CD80 | MCTP2 | FPR1 | VEGFA | TLR6 | PI9 |
| CMPK2 | PLCB1 | MERTK | | LR8 | |
| IDO1 | SIGLEC12 | PLOD2 | | | |
| IL1B | TNFS | SERPINA1 | | | |
| IL3RA | | TIMP1 | | | |
| IL6 | | VCAN | | | |
| SERPING1 | | | | | |
| NFAIP6 | | | | | |

TABLE 4
DEGs of positive and negative control
normalized data at enrollment.

| Gene | P Value | logFC |
|---|---|---|
| EBI3 | 0.005 | 1.887 |
| SYT17 | 0.006 | 1.771 |
| ST8SIA6 | 0.006 | 2.141 |
| IL3RA | 0.007 | 1.830 |
| KDM6B | 0.007 | 1.931 |
| ITK | 0.008 | 1.890 |
| CASP1 | 0.008 | 1.710 |
| NOD2 | 0.008 | 1.848 |
| RIMS2 | 0.008 | 1.669 |
| CD80 | 0.008 | 1.935 |
| PLCb1 | 0.009 | 1.976 |
| NUR77 | 0.009 | 2.137 |
| CD1C | 0.009 | 2.132 |
| CXCL11 | 0.009 | 1.817 |
| TNFRSF1B | 0.010 | 2.273 |
| IFNg | 0.010 | 1.805 |
| VEGFA | 0.011 | 1.767 |
| NCF1B | 0.012 | 1.885 |
| Clorf61 | 0.012 | 1.524 |
| SERPINA1 | 0.013 | 2.324 |
| CD38 | 0.013 | 1.857 |
| TLR2 | 0.013 | 1.925 |
| APOBEC3A | 0.014 | 2.600 |
| MRC1 | 0.014 | 1.729 |
| TAL1 | 0.014 | 1.958 |
| C5AR1 | 0.016 | 2.415 |
| IL1B | 0.017 | 1.775 |
| IDO1 | 0.017 | 1.888 |
| CLEC4E | 0.017 | 2.455 |
| IL6 | 0.018 | 1.904 |
| ABCC8 | 0.018 | 1.573 |
| NLRP3 | 0.019 | 1.664 |
| KCNJ15 | 0.020 | 2.191 |
| PI9 | 0.020 | 2.115 |
| TLR4 | 0.020 | 2.416 |
| LIN7A | 0.021 | 2.180 |
| TLR6 | 0.021 | 2.310 |
| C3ar1 | 0.021 | 2.686 |
| MCTP2 | 0.021 | 1.885 |
| GCH1 | 0.022 | 1.609 |
| CMPK2 | 0.022 | 2.078 |
| TLR9 | 0.022 | 1.571 |
| VCAN | 0.023 | 2.389 |
| TNFRSF1A | 0.023 | 1.744 |
| PDE4B | 0.023 | 2.285 |
| TNFRSF11A | 0.023 | 1.953 |
| MYD88 | 0.024 | 1.698 |
| TLR7 | 0.026 | 2.206 |
| NOTCH1 | 0.026 | 1.554 |
| NCF1 | 0.027 | 2.208 |
| ADORA2A | 0.027 | 1.856 |
| CD163 | 0.027 | 2.163 |
| CCR7 | 0.027 | 1.981 |
| PCSK1 | 0.028 | 1.614 |
| XAf1 | 0.028 | 1.647 |
| TFRC | 0.028 | 1.656 |
| ANKRD22 | 0.029 | 1.915 |
| CLEC4G | 0.029 | 2.565 |
| MERTK | 0.029 | 1.980 |
| APOL1 | 0.030 | 1.607 |
| TNFAIP6 | 0.030 | 1.718 |
| F5 | 0.032 | 2.353 |
| CD300E | 0.032 | 2.628 |
| CFD | 0.033 | 2.189 |
| TGFB1 | 0.033 | 1.709 |
| SNAI3 | 0.034 | 1.829 |
| TIMP1 | 0.035 | 1.871 |
| CH25H | 0.035 | 1.736 |
| TLR1 | 0.037 | 2.242 |
| IL17RB | 0.038 | 1.928 |
| IL32 | 0.038 | 1.589 |
| IL21R | 0.038 | 2.449 |
| ISG15 | 0.038 | 2.387 |
| TLR8 | 0.038 | 2.412 |
| CCL26 | 0.039 | 1.537 |
| PLOD2 | 0.039 | 1.643 |
| WNT5B | 0.040 | 1.712 |
| CCL5 | 0.041 | 1.565 |
| IFI44L | 0.042 | 2.337 |
| SERPING1 | 0.042 | 1.673 |
| FPR1 | 0.044 | 2.284 |
| IFITM1 | 0.047 | 1.910 |
| SIGLEC12 | 0.048 | 2.026 |
| PECAM1 | 0.049 | 2.014 |
| TIMP3 | 0.049 | 1.592 |

TABLE 5
DEGs of raw data at week 4.

| Gene | P value | logFC |
|---|---|---|
| CCL8 | 0.002 | 3.606 |
| WDR66 | 0.004 | 2.873 |
| TNIP3 | 0.005 | 4.542 |
| SIGLEC12 | 0.007 | 3.238 |
| IL10 | 0.010 | 3.532 |
| SERPING1 | 0.011 | 3.874 |
| IL21R | 0.014 | 2.224 |
| CRISPLD2 | 0.015 | 3.838 |
| OLFML3 | 0.015 | 3.132 |
| VCAN | 0.017 | 3.812 |
| TNFAIP6 | 0.019 | 3.824 |
| MERTK | 0.019 | 3.299 |
| MCTP2 | 0.020 | 3.142 |
| KDM5A | 0.020 | 2.778 |
| CFH | 0.020 | 3.361 |
| ANGPT2 | 0.023 | 3.179 |
| CMPK2 | 0.023 | 2.696 |
| FAM110B | 0.024 | 2.557 |
| FCGR2B | 0.025 | 3.697 |
| GXYLT2 | 0.027 | 2.834 |
| PLOD2 | 0.029 | 4.143 |
| IL6 | 0.030 | 4.703 |
| IRF1 | 0.030 | 3.310 |
| HSH2D | 0.030 | 2.620 |
| TNFRSF1B | 0.031 | 4.492 |
| NEK10a | 0.031 | 2.040 |
| HLA-DOA | 0.032 | 2.574 |
| CASP1 | 0.034 | 3.563 |
| HK_GAPDH | 0.035 | 3.129 |
| HK_VPS29 | 0.035 | 2.312 |
| NOTCH1 | 0.036 | 3.245 |
| IL3RA | 0.037 | 3.565 |
| IFITM3 | 0.037 | 3.175 |
| IL1B | 0.039 | 3.854 |
| IGFBP4 | 0.039 | 3.699 |
| CD300E | 0.040 | 3.763 |
| TLR2 | 0.040 | 3.688 |
| IDO1 | 0.041 | 3.462 |
| FPR1 | 0.041 | 3.573 |
| CCNA1 | 0.042 | 1.919 |
| FGF2 | 0.042 | 3.185 |
| PECAM1 | 0.042 | 3.074 |
| CCL18 | 0.043 | 3.505 |
| CLEC4D | 0.043 | 3.319 |
| APOL1 | 0.043 | 2.810 |
| SERPINA1 | 0.044 | 3.422 |
| TLR8 | 0.044 | 3.436 |
| PLCb1 | 0.045 | 2.776 |
| TLR6 | 0.045 | 2.883 |
| PTGS2 | 0.046 | 3.993 |
| HK_UBE2D2 | 0.046 | 3.356 |
| HAPLN3 | 0.048 | 3.012 |
| TIMP1 | 0.048 | 3.871 |
| ADORA2A | 0.048 | 3.603 |
| PI9 | 0.048 | 3.415 |
| CD80 | 0.048 | 3.183 |
| TLR1 | 0.049 | 3.793 |

### Evaluation of Macrophage Specific Genes

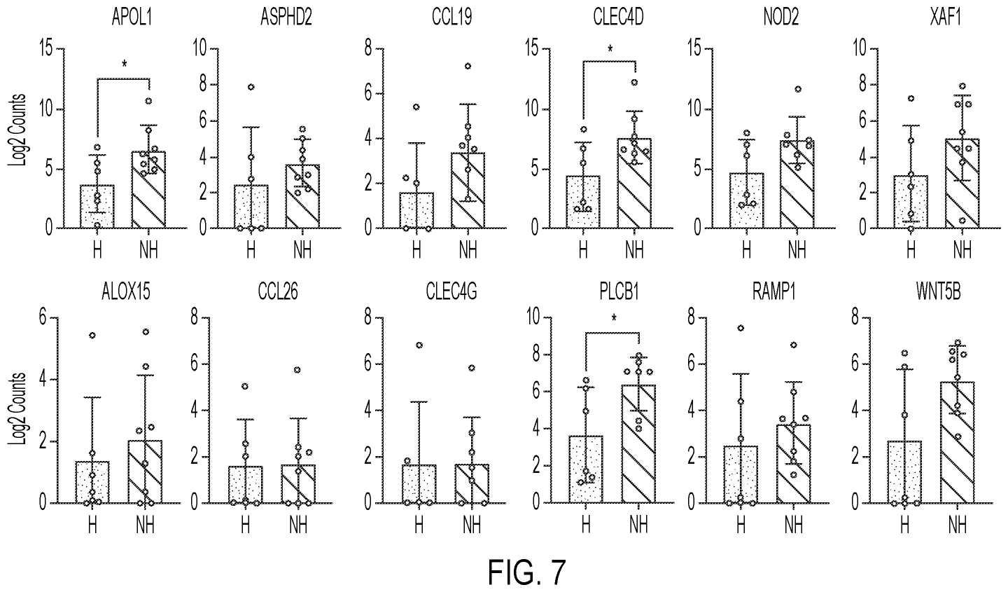

The generally higher expression of inflammation-related genes in non-healing DFUs led to the question that if non-healing DFU tissue contained higher numbers of immune cells, especially macrophages, compared to healing DFUs. Expression of 12 macrophage-specific genes were therefore evaluated. In general, all of these genes were expressed at higher levels in non-healing DFUs compared to healing DFUs at both week 0 and week 4 time points, regardless of whether they were associated with M1 or M2a polarization (FIGS. 2F-2G and 7), although the effect was more pronounced for genes associated with the M1 phenotype (FIG. 2F). These results suggest that the non-healing DFUs contained more macrophages of both phenotypes than healing DFUs.

Gene Level Analyses after Normalization

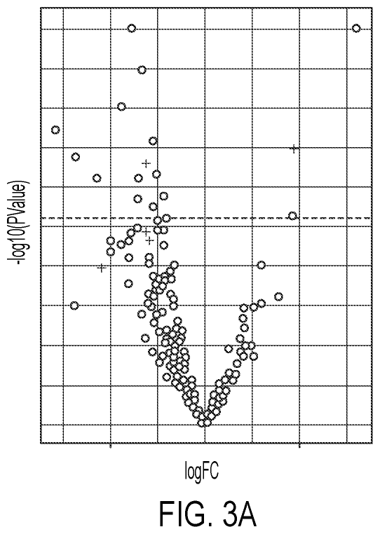

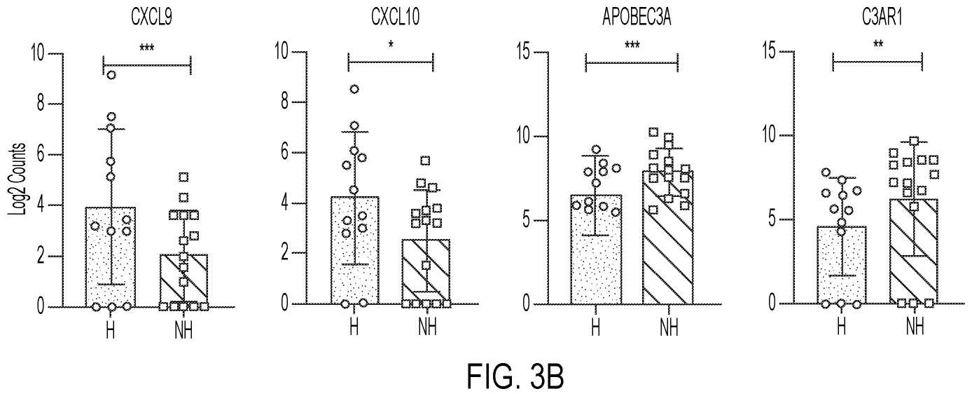

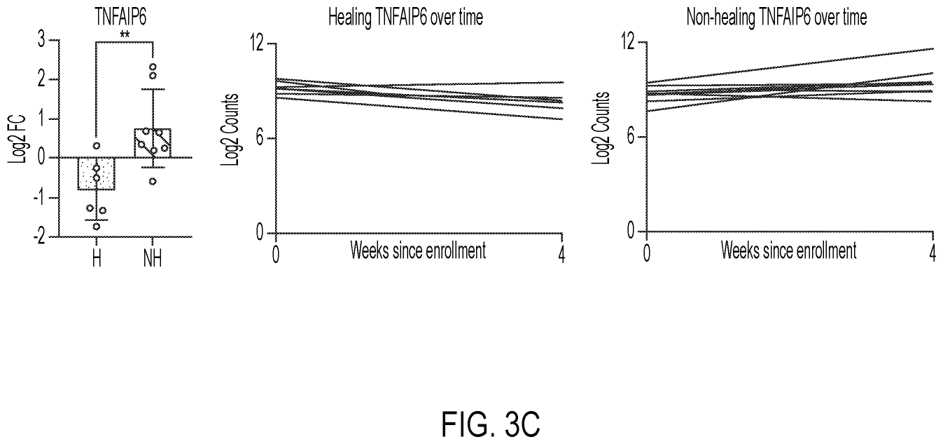

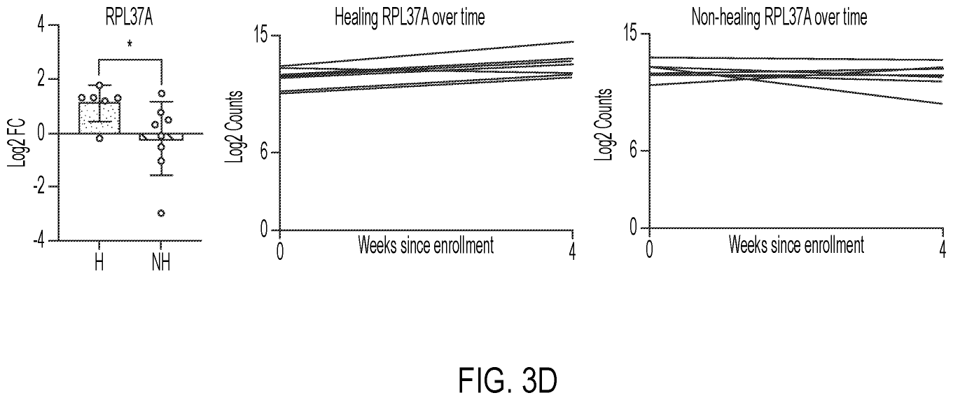

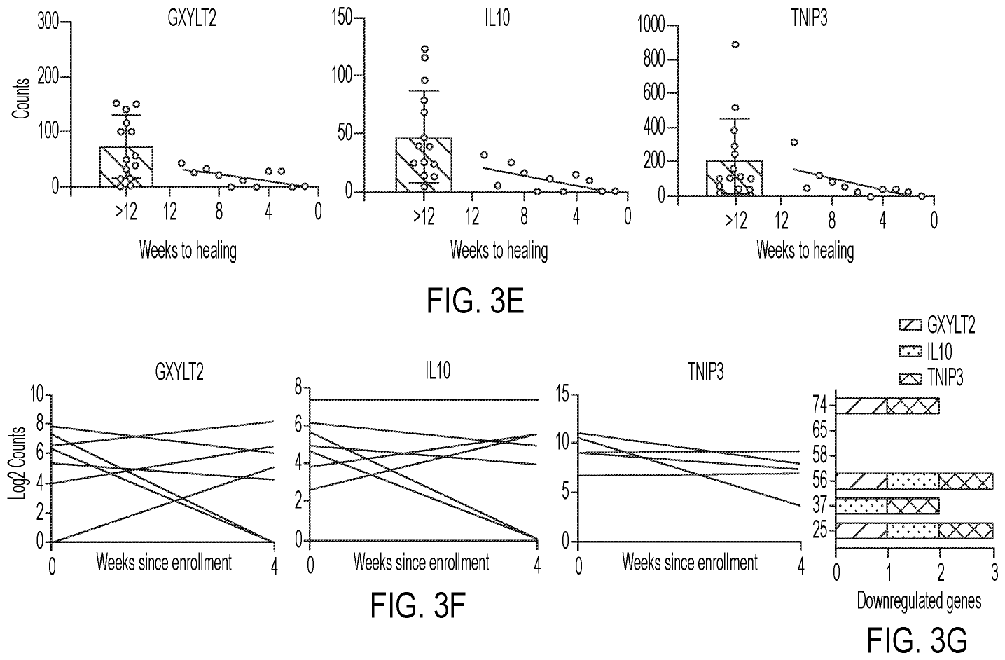

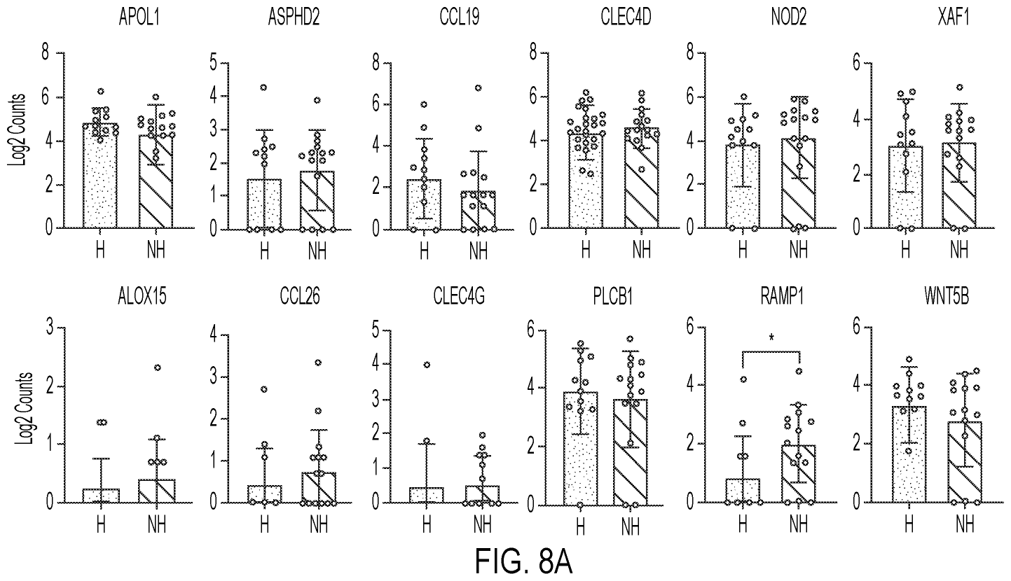

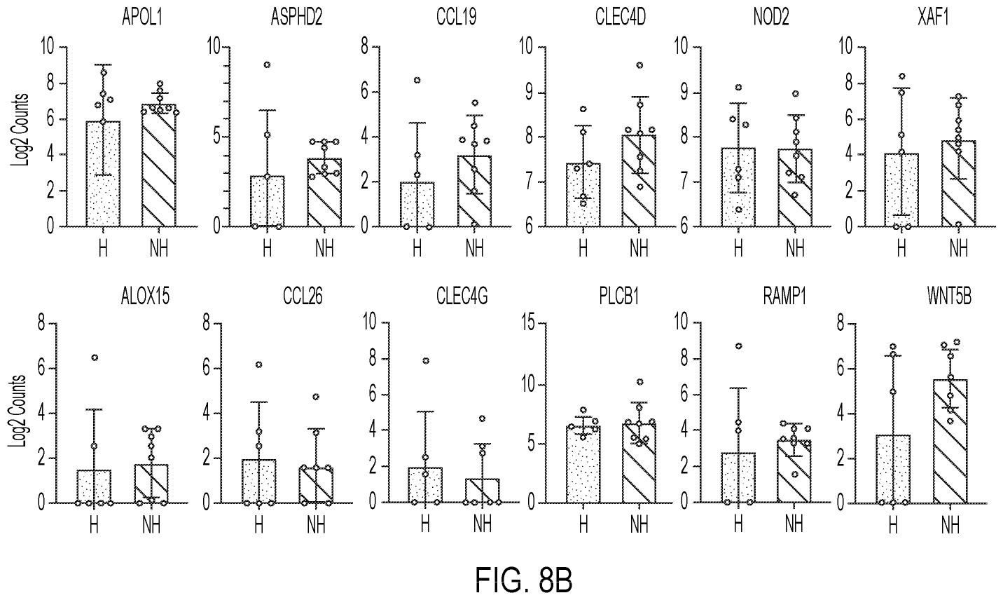

Based on the global increases in gene expression in non-healing DFUs compared to healing DFUs, and especially in macrophage-specific genes, the present study next normalized the data using upper quartile normalization to take these differences in sample composition into account in order to further analyze how cell behavior changes in paired samples over time and how gene expression is influenced by the microbiome. After UQ normalization, macrophage-specific genes were no longer differentially expressed between healing and non-healing DFUs, with the exception of RAMP1, which remained more highly expressed in non-healing DFUs (FIGS. 8A-8B). In fact, only 4 genes were confirmed to be differentially expressed between the groups at p<0.05 and FC>2 (FIG. 3B). Interestingly, the two genes expressed at higher levels in healing DFUs, CXCL9 and CXCL10, are associated with the M1 phenotype of macrophages. The other two DEGs were expressed at higher levels in non-healing DFUs and were associated with M1 polarization (APOBEC3A) and host-microbe communication (C3AR1). When evaluating the change in each gene between week 0 and week 4, only 2 genes were found to change to different extents between groups, and they changed in different directions between healing and non-healing DFUs (FIG. 3C). TNFAIP6, an M1 marker, decreased over time in most of the healing DFUs but increased in most of the non-healing DFUs (FIG. 3C). RPL37A, which was included on the panel as a housekeeping gene and is involved in metabolism, generally increased over time in healing DFUs, whereas it increased in some non-healing DFUs and decreased in others (FIG. 3D).

### Changes in Expression as a Wound Approaches Healing

All of the healing DFUs in this study healed within 12 weeks of the first sample collection, but at different time points, so the present study next investigated whether any gene was associated with the amount of time remaining until complete wound closure across the healing population. It was found that the expression of three genes, GXYLT2, IL10, and TNIP3, was generally lower for DFUs that were closer to healing (FIG. 3E). Moreover, for the DFUs with paired time points at week 0 and week 4, 4 of the 6 patients showed a decrease in at least 2 of these genes over time (FIG. 3G). In contrast, the non-healing DFUs were more variable, with no clear trends compared to healing DFUs.

### Relationships Between Human Gene Expression and Microbial Abundance

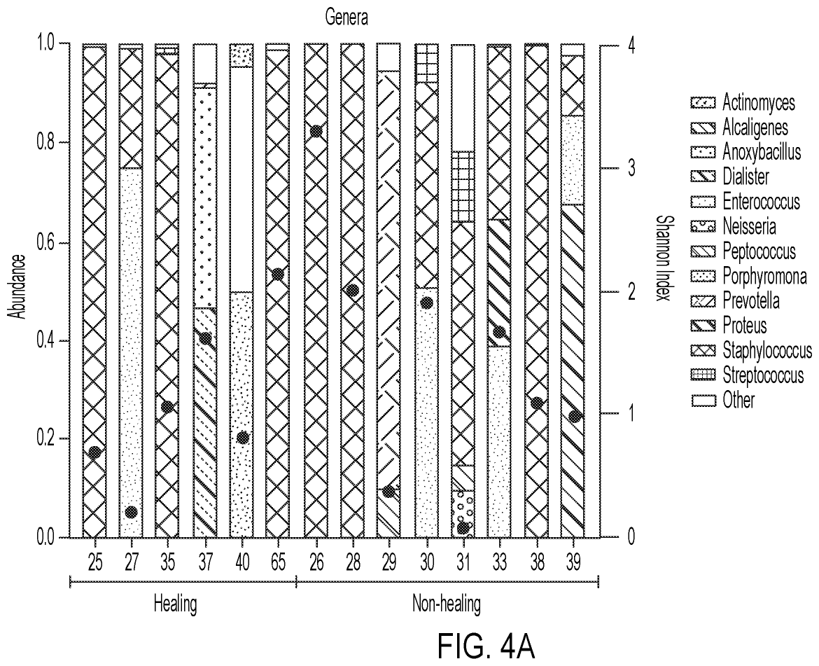

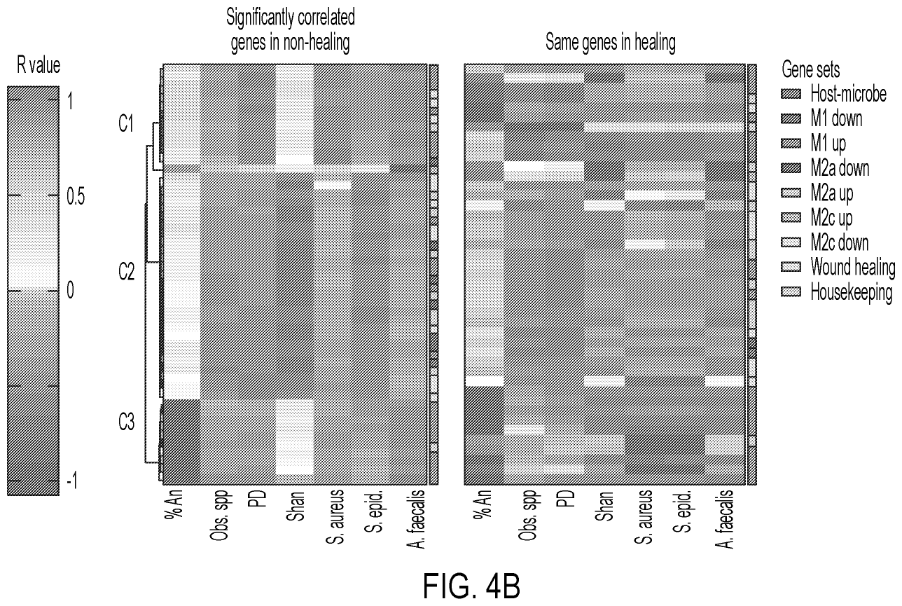

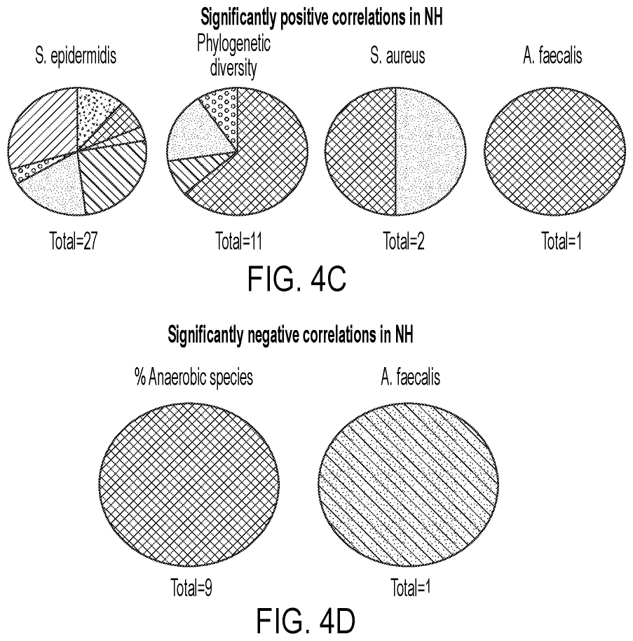

The present study next evaluated how human gene expression was influenced by the microbial communities within the DFUs. There were no obvious trends in the diversity of species or abundance of specific genera between healing and non-healing groups (FIG. 4A). The microbial communities in several subjects were dominated by Staphyloccocus, but differences in patterns between healing and non-healing DFUs were not observed. In the non-healing DFUs, 51 genes were significantly correlated to microbial diversities or a particular species, but no significant correlations were found in the healing DFUs (FIG. 4B). This finding might be partially explained by the lower number of replicates in this group (n=3 in healing compared to n=6 for non-healing), but even when comparing the same genes in non-healing and healing DFUs there were clear difference in patterns of correlation. For example, within the non-healing DFUs, Cluster 1 (C1) comprised genes that were significantly positively correlated to phylogenetic diversity and to S. aureus. These same genes were generally negatively correlated with the same metrics in healing DFUs. Cluster 2 (C2) contained about half of all genes with significant correlations, although no particular gene set was overrepresented in this cluster. In the non-healing DFUs, C2 comprised genes that were positively correlated with S. epidermidis and weakly negatively correlated with the number of observed species, phylogenetic diversity, Shannon index, and abundance of S. aureus. In contrast, in the healing DFUs, these genes were positively correlated with the number of observed species and phylogenetic diversity, and negatively correlated with Shannon index, S. aureus, and S. epidermis. Finally, Cluster 3 (C3) in the non-healing DFUs, which was primarily M1 markers, was negatively correlated with the percentage of anaerobic species and positively correlated to A. faecalis. These trends were similar in healing DFUs.

Across all clusters, 20 of the 51 significantly correlated genes were associated with M1 polarization (FIG. 4C. Interestingly, the gene set with the second greatest number of correlations was housekeeping genes in which S. epidermidis was significantly positively correlated to all 8 of them. Additionally, there were considerably more positive than negative correlations overall and the majority of the genes were correlated to S. epidermidis, which is normally considered a commensal organism (FIG. 4C). Many of these genes were related to regulation of M2a polarization.

### Example 5

The present study highlights several major differences in inflammation-related gene expression between human healing and non-healing DFUs at individual time points and in terms of how they changed in paired samples over time. The present study also found different patterns of correlation with microbial composition. In general, non-healing DFUs expressed higher levels of inflammatory genes at both time points. However, the fact that 6 of 15 non-healing DFUs showed similar or lower levels of inflammation-related gene expression as the healing DFUs highlights substantial heterogeneity in human DFUs, a major difference from murine models. These findings are important because they show that these patients might need to be treated differently depending on the inflammatory state of their wound. For example, highly inflammatory non-healing DFUs can benefit from treatment with products that decrease M1 and/or increase M2 activation of macrophages, such as certain amniotic membrane-derived products, while non-healing DFUs that have low levels of inflammation can be less responsive to these treatments.

The present study also found differences in how genes were regulated over time between healing and non-healing DFUs using paired samples at two time points. Of the 16 genes that were consistently upregulated by non-healing DFUs at both time points, 9 were M1 markers and 7 were M2c markers. Many M1 and M2c markers were associated with the early stages of healing in acute wounds of healthy subjects, so these results are consistent with the assumption that non-healing DFUs fail to transition to later stages of healing. The present study also found that the change in the M1/M2a ratio over time decreased in healing but not non-healing DFUs.

In the present study, it was found that TNFAIP6 was upregulated by non-healing DFUs at both time points.

The present study also showed that the wound microbiome is a regulator of gene expression patterns particularly in non-healing DFUs. Many of the DEGs upregulated by non-healing wounds were related to microbial crosstalk, so the extent of significant correlations to genera and diversity was not particularly surprising. The results show that non-healing DFUs are either more affected by microbes within the wound, or that they contain greater numbers of microbes.

This is the first study to report differences in expression of a large number of genes between human healing and non-healing DFUs, as well as how they change over time and how they correlate with microbiome.

The present study shows that non-healing DFUs generally have higher levels of inflammation that persist over time. The present study also reports differences in crosstalk with microbiome.

### ENUMERATED EMBODIMENTS

In some aspects, the present invention is directed to the following non-limiting embodiments:

Embodiment 1: A method of treating, ameliorating, and/or preventing a wound in a subject in need thereof, the method comprising:
collecting a first sample from the wound at a first timepoint and determining a first CCL1 (C-C Motif Chemokine Ligand 1) level in the first sample;
administering to the subject a first treatment that promotes wound healing;
collecting a second sample from the wound at a second timepoint after the first timepoint and determining a second CCL1 level in the second sample; and
performing one of the following:
   (a) if the second CCL1 level is higher than the first CCL1 level times by a predetermined value, continuing the administration of the first treatment, or
   (b) if the second CCL1 level is equal to or lower than the CCL1 level times by the predetermined value, administering to the subject a second wound healing treatment that is distinct from the first treatment.

Embodiment 2: The method of Embodiment 1, wherein the wound has shown no significant progress toward healing in 15 days prior to the collection of the first sample.

Embodiment 3: The method of any one of Embodiments 1-2, wherein the wound is an ulcer.

Embodiment 4: The method of any one of Embodiments 1-3, wherein the wound is a diabetic ulcer.

Embodiment 5: The method of any one of Embodiments 1-4, wherein collecting the first sample and/or the second sample comprises swabbing the wound, or debriding the wound and collecting the debrided tissue.

Embodiment 6: The method of any one of Embodiments 1-5, wherein the first CCL1 level and/or the second CCL2 level is a mRNA level or a protein level of CCL1.

Embodiment 7: The method of any one of Embodiments 1-6, wherein the first CCL1 level and/or the second CCL2 level is represented as an absolute quantity of CCL1.

Embodiment 8: The method of any one of Embodiments 1-6, wherein the first CCL1 level and/or the second CCL1 level is represented as a ratio between a quantity of CCL1 and a quantity of a reference biomarker.

Embodiment 9: The method of Embodiment 8, wherein the reference biomarker is TNFAIP6 (tumor necrosis factor, alpha-induced protein 6), APOL1 (apolipoprotein L1), or IRF1 (interferon regulatory factor 1).

Embodiment 10: The method of any one of Embodiments 1-9, wherein the first timepoint and the second timepoint are separated by about 5 days to about 10 weeks.

Embodiment 11: The method of any one of Embodiments 1-10, wherein the predetermined value is about 1 or higher.

Embodiment 12: The method of any one of Embodiments 1-11, wherein the first treatment comprises debriding the wound, applying a compression wrapping, applying a compression stocking, applying dressings promoting a moist environment to the wound, applying a wound offloading device, applying a hyperbaric oxygen therapy, applying an antibiotic, or combinations thereof.

Embodiment 13: The method of any one of Embodiments 1-12, wherein the second treatment comprises applying a placental membrane derived construct, applying a bioengineered allogeneic cellular construct, or an immunomodulation medication.

Embodiment 14: The method of any one of Embodiments 1-13, wherein the subject is a mammal, optionally a human.

Embodiment 15: A method of evaluating healing of a wound, the method comprising: collecting a first sample from the wound at a first timepoint and determining a first CCL1 (C-C Motif Chemokine Ligand 1) level in the first sample; and
collecting a second sample from the wound at a second timepoint after the first timepoint and determining a second CCL1 level in the second sample,
wherein
   (a) if the second CCL1 level is higher than the first CCL1 level times by a predetermined value, the healing is significantly progressing, or
   (b) if the second CCL1 level is equal to or lower than the CCL1 level times by the predetermined value, the healing is not significantly progressing.

Embodiment 16: The method of Embodiment 15, wherein the wound has shown no significant progress toward healing in 15 days prior to the collection of the first sample.

Embodiment 17: The method of any one of Embodiments 15-16, wherein the wound is an ulcer.

Embodiment 18: The method of any one of Embodiments 15-17, wherein the wound is a diabetic ulcer.

Embodiment 19: The method of any one of Embodiments 15-18, wherein collecting the first sample and/or the second sample comprises swabbing the wound, or debriding the wound and collecting the debrided tissue.

Embodiment 20: The method of any one of Embodiments 15-19, wherein the first CCL1 level and/or the second CCL2 level is a mRNA level or a protein level of CCL1.

Embodiment 21: The method of any one of Embodiments 15-20, wherein the first CCL1 level and/or the second CCL2 level is represented as an absolute quantity of CCL1.

Embodiment 22: The method of any one of Embodiments 15-20, wherein the first CCL1 level and/or the second CCL1 level is represented as a ratio between a quantity of CCL1 and a quantity of a reference biomarker.

Embodiment 23: The method of Embodiment 22, wherein the reference biomarker is TNFAIP6 (tumor necrosis factor, alpha-induced protein 6), APOL1 (apolipoprotein L1), or IRF1 (interferon regulatory factor 1).

Embodiment 24: The method of any one of Embodiments 15-23, wherein the first timepoint and the second timepoint are separated by about 5 days to about 10 weeks.

Embodiment 25: The method of any one of Embodiment 15-24, wherein the predetermined value is about 1 or higher.

Embodiment 26: The method of any one of Embodiment 15-25, wherein the subject is administered with a treatment for the wound between the collection of the first sample and the collection of the second sample.

Embodiment 27: The method of Embodiment 26, wherein the treatment comprises debriding the wound, applying a compression wrapping, applying a compression stocking, applying dressings promoting a moist environment to the wound, applying a wound offloading device, applying a hyperbaric oxygen therapy, applying an antibiotic, or combinations thereof.

Embodiment 28: The method of any one of Embodiments 26-27, which allows for evaluating efficacy of the treatment.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

1. A method of treating, ameliorating, or preventing a wound in a subject in need thereof, the method comprising:
collecting a first sample from the wound at a first timepoint and determining a first CCL1 (C-C Motif Chemokine Ligand 1) level in the first sample;
administering to the subject a first treatment that promotes wound healing;
collecting a second sample from the wound at a second timepoint after the first timepoint and determining a second CCL1 level in the second sample; and
performing one of the following:
(a) if the second CCL1 level is higher than the first CCL1 level times by a predetermined value, continuing the administration of the first treatment, or
(b) if the second CCL1 level is equal to or lower than the CCL1 level times by the predetermined value, administering to the subject a second wound healing treatment that is distinct from the first treatment.

2. The method of claim 1, wherein at least one of the following applies:
(a) the wound has shown no significant progress toward healing in 15 days prior to the collection of the first sample;
(b) the wound is an ulcer;
(c) the wound is a diabetic ulcer;
(d) collecting the first sample or the second sample comprises swabbing the wound, or debriding the wound and collecting the debrided tissue;
(e) the first CCL1 level or the second CCL2 level is a mRNA level or a protein level of CCL1;
(f) the first timepoint and the second timepoint are separated by about 5 days to about 10 weeks;
(g) the predetermined value is about 1 or higher.

3-6. (canceled)

7. The method of claim 1, wherein the first CCL1 level or the second CCL2 level is represented as an absolute quantity of CCL1.

8. The method of claim 1, wherein the first CCL1 level or the second CCL1 level is represented as a ratio between a quantity of CCL1 and a quantity of a reference biomarker.

9. The method of claim 8, wherein the reference biomarker is TNFAIP6 (tumor necrosis factor, alpha-induced protein 6), APOL1 (apolipoprotein L1), or IRF1 (interferon regulatory factor 1).

10-11. (canceled)

12. The method of claim 1, wherein the first treatment comprises debriding the wound, applying a compression wrapping, applying a compression stocking, applying dressings promoting a moist environment to the wound, applying a wound offloading device, applying a hyperbaric oxygen therapy, applying an antibiotic, or combinations thereof.

13. The method of claim 1, wherein the second treatment comprises applying a placental membrane derived construct, applying a bioengineered allogeneic cellular construct, or an immunomodulation medication.

14. The method of claim 1, wherein the subject is a mammal, or a human.

15. A method of evaluating healing of a wound, the method comprising:
collecting a first sample from the wound at a first timepoint and determining a first CCL1 (C-C Motif Chemokine Ligand 1) level in the first sample; and
collecting a second sample from the wound at a second timepoint after the first timepoint and determining a second CCL1 level in the second sample,
wherein
if the second CCL1 level is higher than the first CCL1 level times by a predetermined value, the healing is significantly progressing, or
(b) if the second CCL1 level is equal to or lower than the CCL1 level times by the predetermined value, the healing is not significantly progressing.

16. The method of claim 15, wherein at least one of the following applies:
(a) the wound has shown no significant progress toward healing in 15 days prior to the collection of the first sample;
(b) the wound is an ulcer;
(c) the wound is a diabetic ulcer;
(d) collecting the first sample or the second sample comprises swabbing the wound, or debriding the wound and collecting the debrided tissue;
(e) the first CCL1 level or the second CCL2 level is a mRNA level or a protein level of CCL1;
(f) the first timepoint and the second timepoint are separated by about 5 days to about 10 weeks;
(g) the predetermined value is about 1 or higher.

17-20. (canceled)

21. The method of claim 15, wherein the first CCL1 level or the second CCL2 level is represented as an absolute quantity of CCL1.

22. The method of claim 15, wherein the first CCL1 level or the second CCL1 level is represented as a ratio between a quantity of CCL1 and a quantity of a reference biomarker.

23. The method of claim 22, wherein the reference biomarker is TNFAIP6 (tumor necrosis factor, alpha-induced protein 6), APOL1 (apolipoprotein L1), or IRF1 (interferon regulatory factor 1).

24-25. (canceled)

26. The method of claim 25, wherein the subject is administered with a treatment for the wound between the collection of the first sample and the collection of the second sample.

27. The method of claim 26, wherein the treatment comprises debriding the wound, applying a compression wrapping, applying a compression stocking, applying dressings promoting a moist environment to the wound, applying a wound offloading device, applying a hyperbaric oxygen therapy, applying an antibiotic, or combinations thereof.

28. The method of claim 26, which allows for evaluating efficacy of the treatment.
